(19) United States
(12) Patent Application Publication
(10) Pub. No.: US 2009/0325993 A1
(43) Pub. Date: Dec. 31, 2009

(54) GLYT1 Transporter Inhibitors and Uses Thereof in Treatment of Neurological and Neuropsychiatric Disorders

(76) Inventors: Nadia Mamoona Ahmad, Surrey (GB); Justine Yeun Quai Lai, Essex (GB); Howard Robert Marshall, Essex (GB); David John Nash, Essex (GB); Roderick Alan Porter, Essex (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 12/524,589

(22) PCT Filed: Jan. 30, 2008
(86) PCT No.: PCT/EP2008/051083

§ 371 Date: Jul. 27, 2009

(30) Foreign Application Priority Data
Feb. 1, 2007 (GB) 0701952.4
Feb. 1, 2007 (GB) 0701978.9

(51) Int. Cl. A61K 31/4439; C07D 235/02; C07D 471/10; A61K 31/4166; A61K 31/4162; A61P 25/18

(52) U.S. Cl. 514/278; 548/300.7; 546/20; 514/385; 514/386

(57) ABSTRACT

Compounds of formula (I) or a salt thereof are provided:

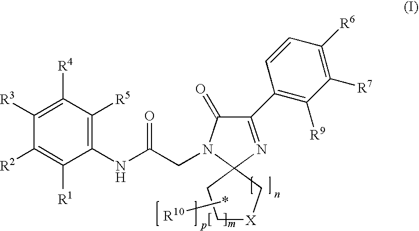

wherein R¹, R², R³, R⁴, R⁵, R⁶, R⁷, R⁹, R¹⁰, X, n, p and m are as defined in the description. Uses of the compounds as medicaments, and in the manufacture of medicament for treating neurological and neuropsychiatric disorders, in particular psychoses, dementia or attention deficit disorder are also disclosed. The invention further discloses pharmaceutical compositions and combinations comprising the compounds.

The present invention relates to compounds, pharmaceutical compositions and medicaments containing them and to their use in treating disorders mediated by GlyT1, including neurological and neuropsychiatric disorders, in particular psychoses, dementia or attention deficit disorder.

Molecular cloning has revealed the existence in mammalian brains of two classes of glycine transporters, termed GlyT1 and GlyT2. GlyT1 is found predominantly in the forebrain and its distribution corresponds to that of glutaminergic pathways and NMDA receptors (Smith, et al., Neuron, 8, 1992: 927-935). Molecular cloning has further revealed the existence of three variants of GlyT1, termed GlyT-la, GlyT-1b and GlyT-1c (Kim et al., Molecular Pharmacology, 45, 1994: 608-617), each of which displays a unique distribution in the brain and peripheral tissues. The variants arise by differential splicing and exon usage, and differ in their N-terminal regions. GlyT2, in contrast, is found predominantly in the brain stem and spinal cord, and its distribution corresponds closely to that of strychnine-sensitive glycine receptors (Liu et al., J. Biological Chemistry, 268, 1993: 22802-22808; Jursky and Nelson, J. Neurochemistry, 64, 1995: 1026-1033). Another distinguishing feature of glycine transport mediated by GlyT2 is that it is not inhibited by sarcosine as is the case for glycine transport mediated by GlyT1. These data are consistent with the view that, by regulating the synaptic levels of glycine, GlyT1 and GlyT2 selectively influence the activity of NMDA receptors and strychnine-sensitive glycine receptors, respectively.

NMDA receptors are critically involved in memory and learning (Rison and Staunton, Neurosci. Biobehav. Rev. 19 533-552 (1995); Danysz et al, Behavioral Pharmacol., 6 455-474 (1995)); and, furthermore, decreased function of NMDA-mediated neurotransmission appears to underlie, or contribute to, the symptoms of schizophrenia (Olney and Farber, Archives General Psychiatry, 52, 998-1007 (1996). Thus, agents that inhibit GlyT1 and thereby increase glycine activation of NMDA receptors can be used as novel antipsychotics and anti-dementia agents, and to treat other diseases in which cognitive processes are impaired, such as attention deficit disorders and organic brain syndromes. Conversely, over-activation of NMDA receptors has been implicated in a number of disease states, in particular the neuronal death associated with stroke and possibly neurodegenerative diseases, such as Alzheimer's disease, multi-infarct dementia, AIDS dementia, Huntington's disease, Parkinson's disease, amyotrophic lateral sclerosis or other conditions in which neuronal cell death occurs, such as stroke or head trauma. Coyle & Puttfarcken, Science, 262, 689-695 (1993); Lipton and Rosenberg, New Engl. J. of Medicine, 330, 613-622 (1993); Choi, Neuron, 1, 623-634 (1988). Thus, pharmacological agents that increase the activity of GlyT1 will result in decreased glycine-activation of NMDA receptors, which activity can be used to treat these and related disease states. Similarly, drugs that directly block the glycine site of the NMDA receptors can be used to treat these and related disease states.

Glycine transport inhibitors are already known in the art, for example as disclosed in published international patent application WO03/055478 (SmithKline Beecham).

However, there still remains the need to identify further compounds that can inhibit GlyT1 transporters, including those that inhibit GlyT1 transporters selectively over GlyT2 transporters.

It has now been found that a novel class of compounds inhibit GlyT1 transporters and are thus of potential use in the treatment of certain neurological and neuropsychiatric disorders, including schizophrenia.

Thus, in the first aspect, there is provided a compound of formula (I) or a salt thereof:

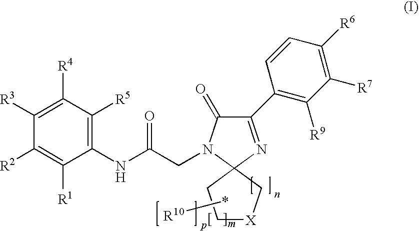

wherein:

R¹ is selected from H, C₁-C₄alkyl, C₁-C₄alkoxy, halo, haloC₁-C₄alkyl, haloC₁-C₄alkoxy, C₁-C₄alkylthio, C₃-C₆cycloalkyl, C₃-C₆cycloalkylC₁-C₄alkyl, C₁-C₄alkylsulfonyl, C₁-C₄alkoxyC₁-C₄alkyl, CONR^{a}R^{b} (wherein R^{a} and R^{b} are independently selected from H and C₁-C₄alkyl, or R^{a} and R^{b}, together with the nitrogen atom to which they are attached, form a 4- to 7-membered ring) and cyano;

R² is selected from H, C₁-C₄alkyl, C₁-C₄alkoxy, halo, haloC₁-C₄alkyl, haloC₁-C₄alkoxy, C₁-C₄alkylthio, C₃-C₆cycloalkyl, C₃-C₆cycloalkylC₁-C₄alkyl, C₁-C₄alkylsulfonyl, C₁-C₄alkoxyC₁-C₄alkyl, CONR^{C}R^{d} (wherein R^{c} and R^{d} are independently selected from H and C₁-C₄alkyl, or R^{c} and R^{d}, together with the nitrogen atom to which they are attached, form a 4- to 7-membered ring) and cyano;

R³ is selected from H, C₁-C₄alkyl, C₁-C₄alkoxy, halo, haloC₁-C₄alkyl, haloC₁-C₄alkoxy, C₁-C₄alkylthio, C₃-C₆cycloalkyl, C₃-C₆cycloalkylC₁-C₄alkyl, C₁-C₄alkylsulfonyl, C₁-C₄alkoxyC₁-C₄alkyl, CONR^{e}R^{f} (wherein R^{e} and R^{f} are independently selected from H and C₁-C₄alkyl, or R^{e} and R^{f}, together with the nitrogen atom to which they are attached, form a 4- to 7-membered ring) and cyano;

or R² and R³ together form a group selected from —O—CH₂—O— and —O—CH₂—CH₂—O—;

R⁴ is selected from H, C₁-C₄alkyl, C₁-C₄alkoxy, halo, haloC₁-C₄alkyl, haloC₁-C₄alkoxy, C₁-C₄alkylthio, C₃-C₆cycloalkyl, C₃-C₆cycloalkylC₁-C₄alkyl, C₁-C₄alkylsulfonyl, C₁-C₄alkoxyC₁-C₄alkyl, CONR^{g}R^{h} (wherein R^{g} and R^{h} are independently selected from H and C₁-C₄alkyl, or R^{g} and R^{h}, together with the nitrogen atom to which they are attached, form a 4- to 7-membered ring) and cyano;

R⁵ is selected from hydrogen, chloro, fluoro, C₁-C₄alkyl and CF₃;

R⁶ is selected from H, C₁-C₄alkyl, C₁-C₄alkoxy, haloC₁-C₄alkyl, haloC₁-C₄alkoxy, halo, cyano, C₁-C₄alkoxyC₁-C₄alkyl and C₁-C₄alkoxyC₁-C₄alkoxy;

R⁷ is selected from H, C₁-C₄alkyl, C₁-C₄alkoxy, haloC₁-C₄alkyl, haloC₁-C₄alkoxy, halo, cyano, C₁₋₄alkoxyC₁₋₄alkyl and C₁-C₄alkoxyC₁-C₄alkoxy;

X is O or NR⁸;

R⁸ is selected from H and C₁-C₃alkyl;

m is selected from 0, 1, 2 and 3, and n is selected from 1, 2 and 3, wherein m+n is 1, 2, 3, 4 or 5;

R⁹ is selected from H and fluoro;

R¹⁰ is independently selected from H and C₁-C₄alkyl; and

p is selected from 1, 2, 3 and 4.

The notations “C_{x-y}” and “Cₓ-C_{y}” are interchangeable.

As used herein, the term “C₁-C₄alkyl” refers to a straight or branched alkyl group containing from 1-4 carbon atoms, in all isomeric forms. Examples include methyl, ethyl, propyl, isopropyl, butyl, isobutyl, sec-butyl and tert-butyl.

As used herein, the term “C₁-C₄alkoxy” refers to the group —O—C₁-C₄alkyl wherein C₁-C₄alkyl is as defined above.

As used herein, the terms “halogen” and its abbreviations “hal” or “halo” refer to fluorine, chlorine, bromine, or iodine.

As used herein, the term “haloC₁-C₄alkyl” refers to a C₁-C₄alkyl group as defined above which is substituted with any number of fluorine, chlorine, bromine, or iodine atoms, including with mixtures of those atoms. A haloalkyl group may, for example contain 1, 2 or 3 halogen atoms. For example, a haloalkyl group may have all hydrogen atoms replaced with halogen atoms. Examples of haloalkyl groups include, but are not limited to, fluoromethyl, difluoromethyl and trifluoromethyl.

As used herein, the term “haloC₁-C₄alkoxy” refers to a C₁-C₄alkoxy group as defined above which is substituted with any number of fluorine, chlorine, bromine, or iodine atoms, including with mixtures of those atoms. A haloalkoxy group may, for example contain 1, 2 or 3 halogen atoms. For example, a haloalkoxy group may have all hydrogen atoms replaced with halogen atoms. Examples of haloalkoxy groups include, but are not limited to, fluoromethyloxy, difluoromethyloxy and trifluoromethyloxy.

As used herein, the term “C₁-C₄alkylsulfonyl” refers to a group —SO₂(C₁-C₄alkyl), wherein C₁-C₄alkyl is as defined above. An example is —SO₂CH₃.

As used herein the term “cyano” refers to a group —CN.

R^{a} and R^{b}, together with the nitrogen atom to which they are attached, may form a saturated 4- to 7-membered ring, ie an azetidinyl, pyrrolidinyl, piperidyl, or azepanyl group. Similarly, R^{c} and R^{d}, R^{e} and R^{f}, R^{g} and R^{h}, Rⁱ and R^{j}, and R^{l} and R^{m} may form such a group within the definition of formula (I) above.

As used herein, the term “C₃-C₆cycloalkyl” refers to a cycloalkyl group consisting of from 3 to 6 carbon atoms, ie cyclopropane, cyclobutane, cyclopentane or cyclohexane.

As used herein, the term “C₁-C₄alkoxyC₁-C₄alkyoxy” refers to the group —OC₁-C₄alkyl-O—C₁-C₄alkyl, wherein C₁-C₄alkyl is as defined above.

As used herein, the term “C₁-C₄alkoxyC₁-C₄alkyl” refers to the group —(C₁-C₄alkyl)-O—(C₁-C₄alkyl), wherein C₁-C₄alkyl is as defined above.

As used herein, the term “C₁-C₄alkylthio” refers to a group —S—(C₁-C₄alkyl). An example is —SCH₃.

In one embodiment R¹ is selected from H, C₁-C₂alkyl, C₁-C₂alkoxy, halo, haloC₁-C₂alkyl, haloC₁-C₂alkoxy, C₁-C₂alkylthio, C₁-C₂alkylsulfonyl, C₁-C₂alkoxyC₁-C₂alkyl and cyano. In a further embodiment, R¹ is selected from H and methyl.

In one embodiment R² is selected from H, C₁-C₂alkyl, C₁-C₂alkoxy, halo, haloC₁-C₂alkyl, haloC₁-C₂alkoxy, C₁-C₂alkylthio, C₁-C₂alkylsulfonyl, C₁-C₂alkoxyC₁-C₂alkyl and cyano. In one embodiment R² is selected from H, halo or halomethyl. In a further embodiment, R² is selected from H, fluoro and trifluoromethyl.

In one embodiment R³ is selected from H, C₁-C₂alkyl, C₁-C₂alkoxy, halo, haloC₁-C₂alkyl, haloC₁-C₂alkoxy, C₁-C₂alkylthio, C₁-C₂alkylsulfonyl, C₁-C₂alkoxyC₁-C₂alkyl and cyano. In one embodiment, R³ is selected from H, methyl, chloro and fluoro. In a further embodiment, R³ is selected from H, methyl and fluoro.

In one embodiment R⁴ is selected from H, C₁-C₂alkyl, C₁-C₂alkoxy, halo, haloC₁-C₂alkyl, haloC₁-C₂alkoxy, C₁-C₂alkylthio, C₁-C₂alkylsulfonyl, C₁-C₂alkoxyC₁-C₂alkyl and cyano. In a further embodiment, R⁴ is selected from H, fluoro and trifluoromethyl.

In one embodiment, R⁵ is H.

In one embodiment, R⁶ is chloro or bromo.

In one embodiment, R⁷ is H.

In one embodiment, X is O.

In one embodiment, X is NR⁸.

In one embodiment, when X is NR⁸, R⁸ is H. In one embodiment, when X is NR⁸, R⁸ is C₁-C₃alkyl. In a further embodiment, R⁸ is methyl.

In one embodiment, R⁹ is H.

In one embodiment R¹⁰ is H. In one embodiment R¹⁰ is C₁-C₄alkyl.

In one embodiment, n is selected from 1 and 2. In a further embodiment, n is 2.

In one embodiment, m is selected from 1 and 2. In a further embodiment, m is 1.

In one embodiment, p is selected from 1 and 2. In a further embodiment, p is 1. In an alternative embodiment p is 2.

In one embodiment, there is provided a compound of formula (Ia) or a salt or solvate thereof:

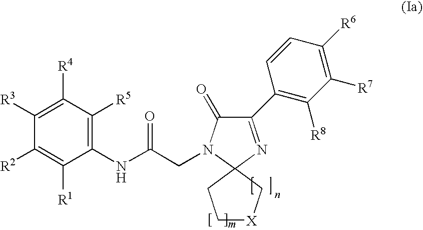

wherein:

R¹ is selected from H, C₁-C₄alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl, CONR^{a}R^{b} (wherein R^{a} and R^{b} are independently selected from H and C₁-C₄alkyl, or R^{a} and R^{b}, together with the nitrogen atom to which they are attached, form a 4- to 7-membered ring) and cyano;

R² is selected from H, C₁-C₄alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl, CONR^{c}R^{d} (wherein R^{c} and R^{d} are independently selected from H and C₁-C₄alkyl, or R^{c} and R^{d}, together with the nitrogen atom to which they are attached, form a 4- to 7-membered ring) and cyano;

R³ is selected from H, C₁₋₄alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl, CONR^{e}R^{f} (wherein R^{e} and R^{f} are independently selected from H and C₁-C₄alkyl, or R^{e} and R^{f}, together with the nitrogen atom to which they are attached, form a 4- to 7-membered ring) and cyano; or R² and R³ together form a group selected from —O—CH₂—O— and —O—CH₂—CH₂—O—;

R⁴ is selected from H, C₁-C₄alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl, CONR^{g}R^{h} (wherein R^{g} and R^{h} are independently selected from H and C₁-C₄alkyl, or R^{g} and R^{h}, together with the nitrogen atom to which they are attached, form a 4- to 7-membered ring) and cyano;

R⁵ is selected from hydrogen, chloro, fluoro, C₁-C₄alkyl and CF₃;

R⁶ is selected from H, C₁-C₄alkyl, C₁-C₄alkoxy, haloC₁-C₄alkyl, haloC₁-C₄alkoxy, halo, cyano, C₁₋₄alkoxyC₁₋₄alkyl and C₁-C₄alkoxyC₁-C₄alkoxy;

R⁷ is selected from H, C₁-C₄alkyl, C₁-C₄alkoxy, haloC₁-C₄alkyl, haloC₁-C₄alkoxy, halo, cyano, C₁₋₄alkoxyC₁₋₄alkyl and C₁-C₄alkoxyC₁-C₄alkoxy;

X is O or NR⁸;

R⁸ is selected from C₁-C₃alkyl;

m is selected from 0, 1, 2 or 3, and n is selected from 1, 2 or 3, wherein m+n is 1, 2, 3, 4 or 5; and

R⁹ is selected from H or fluoro.

In one embodiment there is provided a compound of formula (Ib) or a salt or solvate thereof:

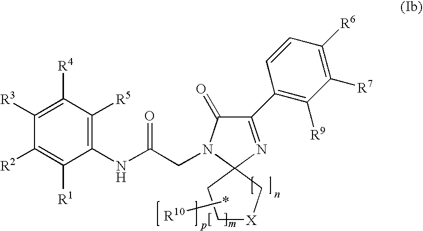

wherein:

R¹ is selected from H, C₁-C₄alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl, CONR^{a}R^{b} (wherein R^{a} and R^{b} are independently selected from H and C₁-C₄alkyl, or R^{a} and R^{b}, together with the nitrogen atom to which they are attached, form a 4- to 7-membered ring) and cyano;

R² is selected from H, C₁-C₄alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl, CONR^{c}R^{d} (wherein R^{c} and R^{d} are independently selected from H and C₁-C₄alkyl, or R^{c} and R^{d}, together with the nitrogen atom to which they are attached, form a 4- to 7-membered ring) and cyano;

R³ is selected from H, C₁₋₄alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl, CONR^{e}R^{f} (wherein R^{e} and R^{f} are independently selected from H and C₁-C₄alkyl, or R^{e} and R^{f}, together with the nitrogen atom to which they are attached, form a 4- to 7-membered ring) and cyano; or R² and R³ together form a group selected from —O—CH₂—O— and —O—CH₂—CH₂—O—;

R⁴ is selected from H, C₁-C₄alkyl, C₁-C₄alkoxy, halo, haloC₁-C₄alkyl, haloC₁-C₄alkoxy, C₁-C₄alkylthio, C₃-C₆cycloalkyl, C₁-C₄alkylsulfonyl, C₁-C₄alkoxyC₁-C₄alkyl, CONR^{g}R^{h} (wherein R^{g} and R^{h} are independently selected from H and C₁-C₄alkyl, or R^{g} and R^{h}, together with the nitrogen atom to which they are attached, form a 4- to 7-membered ring) and cyano;

R⁵ is selected from hydrogen, chloro, fluoro, C₁-C₄alkyl and CF₃;

R⁶ is selected from H, C₁-C₄alkyl, C₁-C₄alkoxy, haloC₁-C₄alkyl, haloC₁-C₄alkoxy, halo, cyano, C₁₋₄alkoxyC₁₋₄alkyl and C₁-C₄alkoxyC₁-C₄alkoxy;

R⁷ is selected from H, C₁-C₄alkyl, C₁-C₄alkoxy, haloC₁-C₄alkyl, haloC₁-C₄alkoxy, halo, cyano, C₁₋₄alkoxyC₁₋₄alkyl and C₁-C₄alkoxyC₁-C₄alkoxy;

X is O or NR⁸;

R⁸ is selected from hydrogen and C₁-C₃alkyl;

R⁹ is selected from H or fluoro.

R¹⁰ is independently selected from C₁₋₄alkyl;

m is selected from 0, 1, 2 or 3, and n is selected from 1, 2 or 3, wherein m+n is 1, 2, 3, 4 or 5; and

p is 1, 2, 3 or 4.

For the avoidance of doubt, the embodiments of any one feature of the compounds of the invention may be combined with any embodiment of another feature of compounds of the invention to create a further embodiment.

Examples of compounds of the invention include:
2-[3-(4-Chlorophenyl)-2-oxo-8-oxa-1,4-diazaspiro[4.5]dec-3-en-1-yl]-N-(3,4-difluorophenyl)acetamide;
2-[3-(4-chlorophenyl)-2-oxo-8-oxa-1,4-diazaspiro[4.5]dec-3-en-1-yl]-N-(2,4-dimethylphenyl)acetamide;
2-[3-(4-Chlorophenyl)-8-methyl-2-oxo-1,4,8-triazaspiro[4.5]dec-3-en-1-yl]-N-(3,4-difluorophenyl)acetamide;
2-[3-(4-Chlorophenyl)-7-methyl-2-oxo-1,4,7-triazaspiro[4.5]dec-3-en-1-yl]-N-(3,4-difluorophenyl)acetamide;
   and salts and solvates thereof.

Further examples of the invention include:
2-[3-(4-Bromophenyl)-2-oxo-7-oxa-1,4-diazaspiro[4.5]dec-3-en-1-yl]-N-[3-(trifluoromethyl)phenyl]acetamide;
2-[3-(4-Bromophenyl)-2-oxo-7-oxa-1,4-diazaspiro[4.4]non-3-en-1-yl]-N-[3-(trifluoromethyl)phenyl]acetamide;
   and salts thereof.

In an embodiment, there is provided a compound of formula (I) as defined above or a pharmaceutically acceptable salt thereof.

Salts of compounds of formula (I) which are suitable for use in medicine are those where the counterion is pharmaceutically acceptable. However, salts having non-pharmaceutically acceptable counterions are within the scope of the present invention, for example, for use as intermediates in the preparation of other compounds of formula (I) and their pharmaceutically acceptable salts.

As used herein, the term “salt” refers to any salt of a compound according to the present invention prepared from an inorganic or organic acid or base, quaternary ammonium salts and internally formed salts. Pharmaceutically acceptable salts are particularly suitable for medical applications because of their greater aqueous solubility relative to the parent compounds. Such salts must clearly have a pharmaceutically acceptable anion or cation. Suitably pharmaceutically acceptable salts of the compounds of the present invention include acid addition salts formed with inorganic acids such as hydrochloric, hydrobromic, hydroiodic, phosphoric, metaphosphoric, nitric and sulfuric acids, and with organic acids, such as tartaric, acetic, trifluoroacetic, citric, malic, lactic, fumaric, benzoic, formic, propionic, glycolic, gluconic, maleic, succinic, (1R)-(−)-10-camphorsulfonic, (1S)-(+)-10-camphorsulfonic, isothionic, mucic, gentisic, isonicotinic, saccharic, glucuronic, furoic, glutamic, ascorbic, anthranilic, salicylic, phenylacetic, mandelic, embonic (pamoic), methanesulfonic, ethanesulfonic, pantothenic, stearic, sulfinilic, alginic, galacturonic and arylsulfonic, for example naphthalene-1,5-disulphonic, naphthalene-1,3-disulphonic, benzenesulfonic and p-toluenesulfonic, acids. Salts having a non-pharmaceutically acceptable anion or cation are within the scope of the invention as useful intermediates for the preparation of physiologically acceptable salts and/or for use in non-therapeutic, for example, in vitro, situations. The salts may have any suitable stoichiometry. For example, a salt may have 1:1 or 2:1 stoichiometry. Non-integral stoichiometry ratios are also possible.

Solvates of the compounds of formula (I) and solvates of the salts of the compounds of formula (I) are included within the scope of the present invention. As used herein, the term “solvate” refers to a complex of variable stoichiometry formed by a solute (in this invention, a compound of formula (I) or a salt thereof) and a solvent. Those skilled in the art of organic chemistry will appreciate that many organic compounds can form such complexes with solvents in which they are reacted or from which they are precipitated or crystallized. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. Most preferably the solvent used is water. Where the solvent used is water such a solvate may then also be referred to as a hydrate.

It will be appreciated by those skilled in the art that certain protected derivatives of compounds of formula (I), which may be made prior to a final deprotection stage, may not possess pharmacological activity as such, but may, in certain instances, be administered orally or parenterally and thereafter metabolised in the body to form compounds of the invention which are pharmacologically active. Such derivatives may therefore be described as “prodrugs”. Further, certain compounds of the invention may be administered as prodrugs. Examples of pro-drug forms for certain compounds of the present invention are described in Drugs of Today, Volume 19, Number 9, 1983, pp 499-538 and in Topics in Chemistry, Chapter 31, pp 306-316 and in “Design of Prodrugs” by H. Bundgaard, Elsevier, 1985, Chapter 1 (the disclosures in which documents are incorporated herein by reference). It will further be appreciated by those skilled in the art, that certain moieties, known to those skilled in the art as “pro-moieties”, for example as described by H. Bundgaard in “Design of Prodrugs” (the disclosure in which document is incorporated herein by reference) may be placed on appropriate functionalities when such functionalities are present within compounds of the invention. Examples of prodrugs for certain compounds of the invention include: esters, carbonate esters, hemi-esters, phosphate esters, nitro esters, sulfate esters, sulfoxides, amides, carbamates, azo-compounds, phosphamides, glycosides, ethers, acetals and ketals.

Hereinafter, compounds of formula (I) (whether in solvated or unsolvated form) or their pharmaceutically acceptable salts (whether in solvated or unsolvated form) or prodrugs thereof defined in any aspect of the invention (except intermediate compounds in chemical processes) are referred to as “compounds of the invention”.

The compounds of formula (I) may have the ability to crystallise in more than one form. This is a characteristic known as polymorphism, and it is understood that such polymorphic forms (“polymorphs”) are within the scope of formula (I). Polymorphism generally can occur as a response to changes in temperature or pressure or both and can also result from variations in the crystallisation process. Polymorphs can be distinguished by various physical characteristics known in the art such as x-ray diffraction patterns, solubility, and melting point.

Certain of the compounds described herein may exist in stereoisomeric forms (i.e. they may contain one or more asymmetric carbon atoms or may exhibit cis-trans isomerism), for example when p in formula (I) is 1 and R¹⁰ is not H; or if one of R¹-R⁵ contains an asymmetric carbon. The individual stereoisomers (enantiomers and diastereoisomers) and mixtures of these are included within the scope of the present invention. Stereoisomers may be separated by high-performance liquid chromatography or other appropriate means. When a compound is desired as a single enantiomer, it may be obtained by stereospecific synthesis or by resolution of the final product or any convenient intermediate. Resolution of the final product, an intermediate, or a starting material may be effected by any suitable method known in the art. See, for example, Stereochemistry of Organic Compounds by E. L. Eliel, S. H. Wilen, and L. N. Mander (Wiley-Interscience, 1994). Likewise, it is understood that compounds of formula (I) may exist in tautomeric forms other than that shown in the formula and these are also included within the scope of the present invention.

In one embodiment, an optically pure enantiomer of a compound of the present invention is provided. The term “optically pure enantiomer” means that the compound contains greater than about 90% of the desired isomer by weight, such as greater than about 95% of the desired isomer by weight, and or greater than about 99% of the desired isomer by weight, said weight percent based upon the total weight of the isomer(s) of the compound.

Compounds of general formula (I) may be prepared by methods known in the art of organic synthesis as set forth in part by the following synthesis schemes. It is also recognised that in all of the schemes described below, it is well understood that protecting groups for sensitive or reactive groups are employed where necessary in accordance with general principles of chemistry. Protecting groups are manipulated according to standard methods of organic synthesis (T. W. Greene and P. G. M. Wuts (1991) Protecting Groups in Organic Synthesis, John Wiley & Sons). These groups are removed at a convenient stage of the compound synthesis using methods that are readily apparent to those skilled in the art. The selection of processes as well as the reaction conditions and order of their execution shall be consistent with the preparation of compounds of formula (I).

Typical reaction routes for the preparation of a compound of formula (I) as hereinbefore defined, are shown below. Unless otherwise stated, substituents are as defined for formula (I) hereinabove:

Compounds of formula (I) can be prepared by reacting a compound of formula (II) with a base, for example sodium hydride, in a suitable inert solvent, for example dimethylformamide, followed by treatment with a compound of formula (III) where Z is halogen, for example chloro, as shown in Scheme 1.

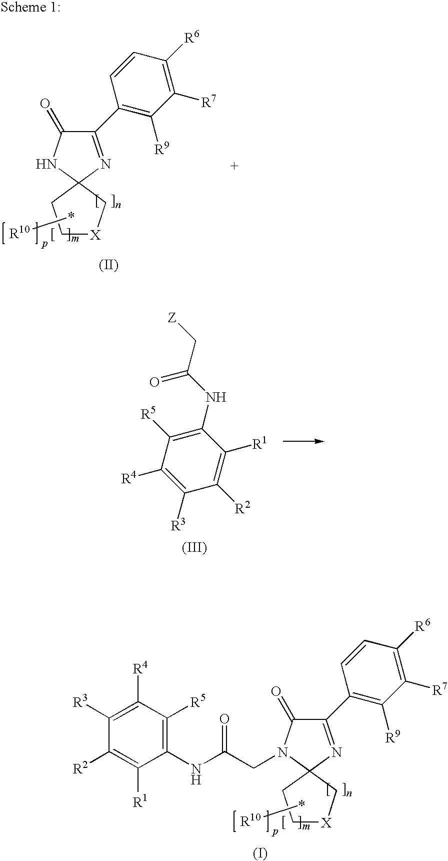

Compounds of formula (III) can be prepared by standard methods, for example as shown in Scheme 2. For example, an aniline of formula (IV) may be combined with an haloacetyl halide of formula (XV) where Z and Z′ are halogen, for example chloroacetyl chloride or bromoacetyl chloride in an inert solvent, for example, dioxan and heated to give a compound of formula (III).

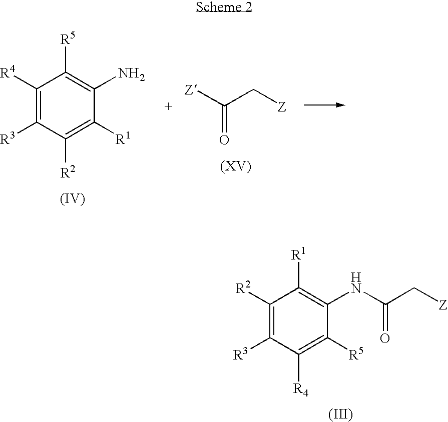

Compounds of formula (II) may be prepared by desulphurisation of compounds of formula (V) using an oxidising agent, for example hydrogen peroxide as shown for example in Scheme 3.

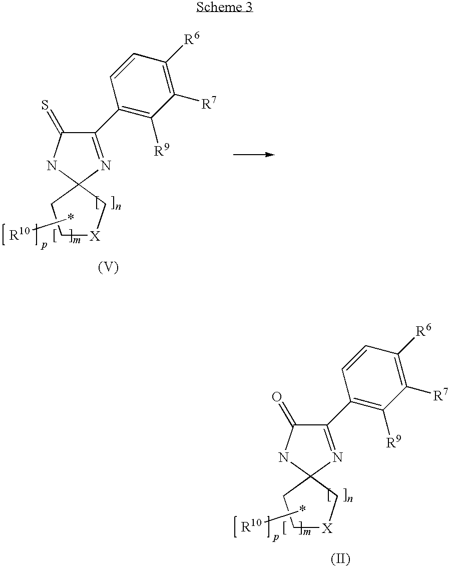

Compounds of formula (V) can be prepared by treating a ketothioamide of formula (VI) with the appropriate ketone of formula (VII) in the presence of a source of ammonia, for example ammonium acetate as shown in Scheme 4. In one embodiment, this reaction is performed in a solvent, for example isopropanol, at room or elevated temperature, preferably elevated temperature, for example at reflux.

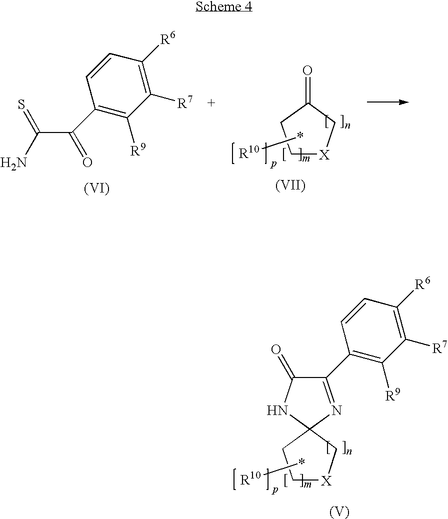

Thioamides of formula (VI) can be prepared from acylnitriles of formula (VIII) by treating with, for example hydrogen sulphide in the presence of an organic base, for example triethylamine in an inert solvent, for example diethyl ether at room temperature. Acylnitriles of formula (VIII) can be prepared from the appropriate acid chloride (IX) and a source of cyanide, conveniently copper (I) cyanide, at elevated temperatures, for example greater than 150° C. preferably in the absence of solvent.

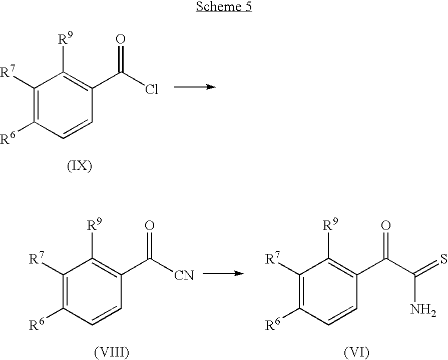

Alternatively, compounds of formula (II) can be synthesised as shown in Scheme 6.

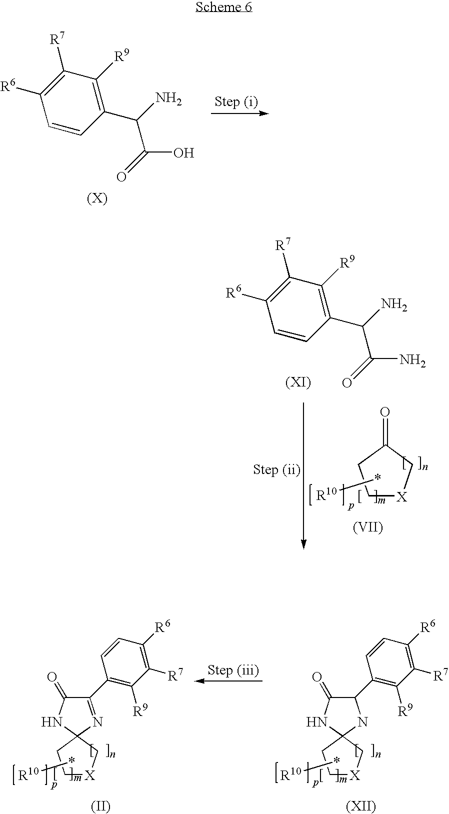

The arylglycine of formula (X) can be converted, step (i), to the corresponding arylglycinamide of formula (XI) by standard methods, for example, by reaction of compounds of formula (X) with thionyl chloride or acetyl chloride in methanol, followed by subsequent reaction of the intermediate methyl ester hydrochloride with aqueous ammonia.

Arylglycinamides of formula (XI) can be converted to compounds of formula (XII), step (ii), by condensation with ketones of formula (VII), for example, by heating in an inert solvent such as methanol, in the presence or absence of a catalyst such as H—Y zeolites.

Oxidation of compounds of formula (XII), step (iii), to afford compounds of formula (II) can be achieved by methods known in the art, for example, by reaction with N-bromosuccinimide in an inert solvent, such as dichloromethane.

Compounds of formula (II) can also be converted to compounds of formula (I) as shown in Scheme 7.

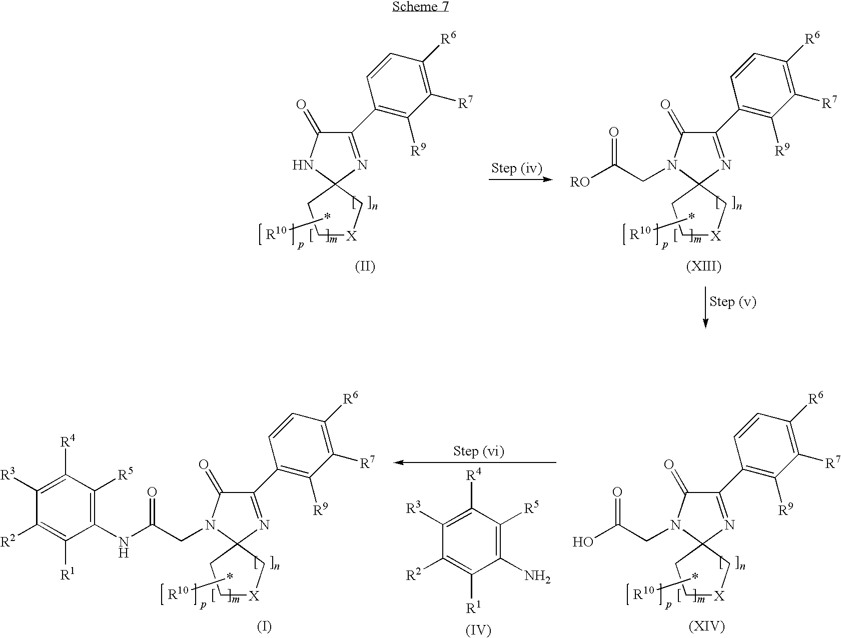

Compounds of formula (XIII) can be prepared using standard methods from compounds of formula (II), step (iv), for example, by reaction with an appropriate alkyl haloacetate ZR′, in which Z is halogen and R′ is an alkyl acetate group for example ethyl bromoacetate, in the presence of a base, such as sodium hydride or potassium carbonate, in a suitable inert solvent, such as dimethylformamide, at room temperature or elevated temperature as appropriate.

Removal of the ester group R from compounds of formula (XIII) to afford the acids of formula (XIV), step (v), can be achieved by known methods, for example by use of a base, such as sodium hydroxide, in an inert solvent, such as aqueous methanol or aqueous ethanol, with or without heating as appropriate.

Compounds of formula (XIV) can be converted to compounds of formula (I), step (vi), by reaction with an aniline of formula (IV) using a variety of methods known in the art. For example, the acylation step (vi) can be achieved by reaction of the acid (XIV) with an aniline of formula (IV), in an inert solvent, such as dichloromethane in the presence of a coupling reagent, for example a diimide reagent such as N,N dicyclohexylcarbodiimide (DCC), N-(3-(dimethylamino)propyl)-N-ethylcarbodiimide hydrochloride (EDC), or O-(7-azabenzotriazol-1-yl)-1,1,3,3-tetramethyluronium hexafluoro phosphate (HATU). Alternatively, compounds of formula (XIV) are converted to compounds of formula (XVI)

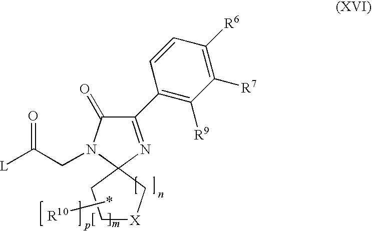

wherein L represents a suitable leaving group. Examples of leaving groups include halogen, OC(═O)alkyl, OC(═O)O-alkyl and OSO₂Me. Compounds of formula (XVI) in which, for example, L is halogen, may be converted to compounds of formula (I) by acylation step (vi), which may be carried out in an inert solvent such as dichloromethane, in the presence of a base, such as triethylamine.

Within the schemes there is scope to convert a group R¹ into another group R¹ and similarly for groups R², R³, R⁴, R⁵, R⁶, R⁷, and R⁹.

Within the schemes X is defined as in formula (I) or within the schemes X may be a group NP where P is a suitable protecting group such as a urethane, for example, tert-butoxycarbonyl or benzyloxycarbonyl; an amide such as acetyl or an optionally substituted N-benzyl for example 4-methoxybenzyl.

Within the schemes, when X is NP as herein defined, compounds may be converted to compounds where X is NR^{8′} and R^{8′} is H, by standard deprotection methods. For example where P is tert-butoxycarbonyl, compounds may be treated with an acid such as trifluoroacetic acid in dichloromethane or hydrogen chloride in dioxan to provide compounds in which X is NH. Compounds in which X is NH may be converted to compounds in which X is NR⁸ and R⁸ is C₁-C₃alkyl by standard N-alkylation methods. For example, treatment of a compound in which X is NH with an aldehyde such as formaldehyde in the presence of a reducing agent such as sodium cyanoborohydride or sodium triacetoxyborohydride in a solvent such as methanol will provide a compound where X is NR⁸ and R⁸ is methyl. Alternatively treatment of a compound in which X is NH with an alkylating agent such as iodoethane in a solvent such as dimethylformamide or tetrahydrofuran, optionally in the presence of a base such as sodium carbonate will provide a compound where X is NR⁸ and R⁸ is ethyl.

Compounds of formula (I) can be converted into further compounds of formula (I) using standard techniques.

Compounds of formula (IV), (VII), (IX), (X) and (XV) are commercially available or may be prepared by standard techniques known in the art.

Salts may be prepared conventionally by reaction with the appropriate acid or acid derivative.

The compounds of the present invention inhibit the GlyT1 transporter as measured by the assay below. Such compounds are therefore of potential utility for the treatment of certain neurological and neuropsychiatric disorders. The compounds may selectively inhibit the GlyT1 transporter over the GlyT2 transporter. Some compounds of the invention may have mixed GlyT1/GlyT2 activity.

The affinities of the compounds of this invention for the GlyT1 transporter can be determined by the following assay. In the assays used herein, the compounds of the present invention were not necessarily from the same batch described above. The test compound made in one batch may have been combined with other batch(es) for the assay(s).

HEK293 cells expressing the Glycine (Type 1) transporter were grown in cell culture medium [DMEM/NUT mix F12 containing 2 mM L-Glutamine, 0.8 mg/mL G418 and 10% heat inactivated fetal calf serum] at 37° C. and 5% CO₂. Cells grown to 70-80% confluency in T175 flasks were harvested and resuspended at 4×10⁵ cells/mL in assay buffer [140 mM NaCl, 5.4 mM KCl, 1.8 mM CaCl₂, 0.8 mM MgSO₄, 20 mM HEPES, 5 mM glucose and 5 mM alanine, pH 7.4]. Compounds were serially diluted 2.5-fold in DMSO from a top concentration of 2.5 mM with each compound giving a 11 data point dose-response. 100 nL of compound at each concentration was added to the assay plate. An equal volume of Leadseeker™ WGA SPA beads (12.5 mg/ml suspended in assay buffer) was added to the cell suspension and 5 μL of the cell/bead suspension transferred to each well of a 384-well white solid bottom plate (1,000 cells/well) containing 100 nL of test compounds. Substrate (5 μL) was added to each well [1:100 dilution of [³H]-glycine stock in assay buffer containing 2.5 μM glycine). Final DMSO concentration was 1% v/v. Data was collected using a Perkin Elmer Viewlux. plC₅₀ values were determined using ActivityBase.

Compounds are considered to have activity at the GlyT1 transporter if they have a plC₅₀ of 5.0 or above. The example compounds below and the individually named compounds above were found to have an average plC₅₀ at the GlyT1 transporter of 5.9 or above. Advantageously, compounds of the invention may have a plC₅₀ at the GlyT1 transporter of greater than 7.0.

As used herein, the term “a disorder mediated by GlyT1” refers to a disorder that may be treated by the administration of a medicament that alters the activity of the GlyT1 transporter. The disorders mediated by GlyT1 referred to herein include neurological and neuropsychiatric disorders, including psychoses such as schizophrenia, dementia and other forms of impaired cognition such as attention deficit disorders and organic brain syndromes. Other neuropsychiatric disorders include drug-induced (phencyclidine, ketamine and other dissociative anesthetics, amphetamine and other psychostimulants and cocaine) psychosis, psychosis associated with affective disorders, brief reactive psychosis, schizoaffective psychosis, and psychosis NOS, “schizophrenia-spectrum” disorders such as schizoid or schizotypal personality disorders, or illness associated with psychosis (such as major depression, manic depressive (bipolar) disorder, Alzheimer's disease and post-traumatic stress syndrome), and NMDA receptor-related disorders such as autism, depression, benign forgetfulness, childhood learning disorders and closed head injury. Other disorders include Parkinson's disease, dyskinetic disorders, cognitive impairment, emesis, movement disorders, amnesia, circadian rhythm disorders, aggression and vertigo.

Accordingly, in one aspect of the invention, there is provided a compound of formula (I) as hereinbefore described or a salt or solvate thereof, for use as a medicament. In a further aspect of the invention, there is provided a compound of formula (I) or a salt thereof, for use in the treatment of a disorder mediated by GlyT1.

In order to use a compound of the present invention as a medicament, it will normally be formulated into a pharmaceutical composition in accordance with standard pharmaceutical practice. The present invention also provides a pharmaceutical composition, which comprises a compound of formula (I) or a salt or solvate thereof, and a carrier, diluent or excipient.

In a further aspect, the present invention provides a process for preparing a pharmaceutical composition, the process comprising mixing a compound of formula (I) or a salt or solvate thereof and a carrier, diluent or excipient.

In another aspect of the invention, there is provided a method of treating a mammal, including a human, suffering from or susceptible to a disorder mediated by GlyT1, which comprises administering an effective amount of a compound of formula (I) as hereinbefore defined or a salt thereof.

In another aspect of the invention, there is provided use of a compound of formula (I) as hereinbefore defined or a salt thereof in the preparation of a medicament for the treatment of a disorder mediated by GlyT1.

In one embodiment, the disorder mediated by GlyT1 to be treated by the use or method as hereinbefore described is a psychosis, including schizophrenia, dementia and attention deficit disorders. In one embodiment, the disorder is schizophrenia.

As used herein, the term “effective amount” means that amount of a drug or pharmaceutical agent that will elicit the biological or medical response of a tissue, system, animal or human that is being sought, for instance, by a researcher or clinician.

Within the context of the present invention, the terms used herein are classified in the Diagnostic and Statistical Manual of Mental Disorders, 4^{th} Edition, published by the American Psychiatric Association (DSM-IV) and/or the International Classification of Diseases, 10^{th} Edition (ICD-10). The various subtypes of the disorders mentioned herein are contemplated as part of the present invention. Numbers in brackets after the listed diseases below refer to the classification code in DSM-IV.

In particular, the compounds of the invention may be of use in the treatment of schizophrenia including the subtypes Paranoid Type (295.30), Disorganised Type (295.10), Catatonic Type (295.20), Undifferentiated Type (295.90) and Residual Type (295.60); Schizophreniform Disorder (295.40); Schizoaffective Disorder (295.70) including the subtypes Bipolar Type and Depressive Type; Delusional Disorder (297.1) including the subtypes Erotomanic Type, Grandiose Type, Jealous Type, Persecutory Type, Somatic Type, Mixed Type and Unspecified Type; Brief Psychotic Disorder (298.8); Shared Psychotic Disorder (297.3); Psychotic Disorder Due to a General Medical Condition including the subtypes With Delusions and With Hallucinations; Substance-Induced Psychotic Disorder including the subtypes With Delusions (293.81) and With Hallucinations (293.82); and Psychotic Disorder Not Otherwise Specified (298.9).

The compounds of the invention may also be of use in the treatment of mood disorders including Major Depressive Episode, Manic Episode, Mixed Episode and Hypomanic Episode; Depressive Disorders including Major Depressive Disorder, Dysthymic Disorder (300.4), Depressive Disorder Not Otherwise Specified (311); Bipolar Disorders including Bipolar I Disorder, Bipolar II Disorder (Recurrent Major Depressive Episodes with Hypomanic Episodes) (296.89), Cyclothymic Disorder (301.13) and Bipolar Disorder Not Otherwise Specified (296.80); Other Mood Disorders including Mood Disorder Due to a General Medical Condition (293.83) which includes the subtypes With Depressive Features, With Major Depressive-like Episode, With Manic Features and With Mixed Features), Substance-Induced Mood Disorder (including the subtypes With Depressive Features, With Manic Features and With Mixed Features) and Mood Disorder Not Otherwise Specified (296.90).

The compounds of the invention may also be of use in the treatment of anxiety disorders including Panic Attack, Agoraphobia, Panic Disorder, Agoraphobia Without History of Panic Disorder (300.22), Specific Phobia (300.29) including the subtypes Animal Type, Natural Environment Type, Blood-Injection-Injury Type, Situational Type and Other Type), Social Phobia (300.23), Obsessive-Compulsive Disorder (300.3), Posttraumatic Stress Disorder (309.81), Acute Stress Disorder (308.3), Generalized Anxiety Disorder (300.02), Anxiety Disorder Due to a General Medical Condition (293.84), Substance-Induced Anxiety Disorder and Anxiety Disorder Not Otherwise Specified (300.00).

The compounds of the invention may also be of use in the treatment of substance-related disorders including Substance Use Disorders such as Substance Dependence and Substance Abuse; Substance-Induced Disorders such as Substance Intoxication, Substance Withdrawal, Substance-Induced Delirium, Substance-Induced Persisting Dementia, Substance-Induced Persisting Amnestic Disorder, Substance-Induced Psychotic Disorder, Substance-Induced Mood Disorder, Substance-Induced Anxiety Disorder, Substance-Induced Sexual Dysfunction, Substance-Induced Sleep Disorder and Hallucinogen Persisting Perception Disorder (Flashbacks); Alcohol-Related Disorders such as Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-Induced Persisting Dementia, Alcohol-Induced Persisting Amnestic Disorder, Alcohol-Induced Psychotic Disorder, Alcohol-Induced Mood Disorder, Alcohol-Induced Anxiety Disorder, Alcohol-Induced Sexual Dysfunction, Alcohol-Induced Sleep Disorder and Alcohol-Related Disorder Not Otherwise Specified (291.9); Amphetamine (or Amphetamine-Like)-Related Disorders such as Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-Induced Mood Disorder, Amphetamine-Induced Anxiety Disorder, Amphetamine-Induced Sexual Dysfunction, Amphetamine-Induced Sleep Disorder and Amphetamine-Related Disorder Not Otherwise Specified (292.9); Caffeine Related Disorders such as Caffeine Intoxication (305.90), Caffeine-Induced Anxiety Disorder, Caffeine-Induced Sleep Disorder and Caffeine-Related Disorder Not Otherwise Specified (292.9); Cannabis-Related Disorders such as Cannabis Dependence (304.30), Cannabis Abuse (305.20), Cannabis Intoxication (292.89), Cannabis Intoxication Delirium, Cannabis-Induced Psychotic Disorder, Cannabis-Induced Anxiety Disorder and Cannabis-Related Disorder Not Otherwise Specified (292.9); Cocaine-Related Disorders such as Cocaine Dependence (304.20), Cocaine Abuse (305.60), Cocaine Intoxication (292.89), Cocaine Withdrawal (292.0), Cocaine Intoxication Delirium, Cocaine-Induced Psychotic Disorder, Cocaine-Induced Mood Disorder, Cocaine-Induced Anxiety Disorder, Cocaine-Induced Sexual Dysfunction, Cocaine-Induced Sleep Disorder and Cocaine-Related Disorder Not Otherwise Specified (292.9); Hallucinogen-Related Disorders such as Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-Induced Psychotic Disorder, Hallucinogen-Induced Mood Disorder, Hallucinogen-Induced Anxiety Disorder and Hallucinogen-Related Disorder Not Otherwise Specified (292.9); Inhalant-Related Disorders such as Inhalant Dependence (304.60), Inhalant Abuse (305.90), Inhalant Intoxication (292.89), Inhalant Intoxication Delirium, Inhalant-Induced Persisting Dementia, Inhalant-Induced Psychotic Disorder, Inhalant-Induced Mood Disorder, Inhalant-Induced Anxiety Disorder and Inhalant-Related Disorder Not Otherwise Specified (292.9); Nicotine-Related Disorders such as Nicotine Dependence (305.1), Nicotine Withdrawal (292.0) and Nicotine-Related Disorder Not Otherwise Specified (292.9); Opioid-Related Disorders such as Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9); Phencyclidine (or Phencyclidine-Like)-Related Disorders such as Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9); Sedative-, Hypnotic-, or Anxiolytic-Related Disorders such as Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic-Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-Induced Anxiety Disorder Sedative-, Hypnotic-, or Anxiolytic-Induced Sexual Dysfunction, Sedative-, Hypnotic-, or Anxiolytic-Induced Sleep Disorder and Sedative-, Hypnotic-, or Anxiolytic-Related Disorder Not Otherwise Specified (292.9); Polysubstance-Related Disorder such as Polysubstance Dependence (304.80); and Other (or Unknown) Substance-Related Disorders such as Anabolic Steroids, Nitrate Inhalants and Nitrous Oxide.

The compounds of the invention may also be of use in the treatment of sleep disorders including primary sleep disorders such as Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition; and Substance-Induced Sleep Disorder including the subtypes Insomnia Type, Hypersomnia Type, Parasomnia Type and Mixed Type.

The compounds of the invention may also be of use in the treatment of eating disorders such as Anorexia Nervosa (307.1) including the subtypes Restricting Type and Binge-Eating/Purging Type; Bulimia Nervosa (307.51) including the subtypes Purging Type and Nonpurging Type; Obesity; Compulsive Eating Disorder; and Eating Disorder Not Otherwise Specified (307.50).

The compounds of the invention may also be of use in the treatment of Autistic Disorder (299.00); Attention-Deficit/Hyperactivity Disorder including the subtypes Attention-Deficit/Hyperactivity Disorder Combined Type (314.01), Attention-Deficit/Hyperactivity Disorder Predominantly Inattentive Type (314.00), Attention-Deficit/Hyperactivity Disorder Hyperactive-Impulse Type (314.01) and Attention-Deficit/Hyperactivity Disorder Not Otherwise Specified (314.9); Hyperkinetic Disorder; Disruptive Behaviour Disorders such as Conduct Disorder including the subtypes childhood-onset type (321.81), Adolescent-Onset Type (312.82) and Unspecified Onset (312.89), Oppositional Defiant Disorder (313.81) and Disruptive Behaviour Disorder Not Otherwise Specified; and Tic Disorders such as Tourette's Disorder (307.23).

The compounds of the invention may also be of use in the treatment of Personality Disorders including the subtypes Paranoid Personality Disorder (301.0), Schizoid Personality Disorder (301.20), Schizotypal Personality Disorder (301.22), Antisocial Personality Disorder (301.7), Borderline Personality Disorder (301.83), Histrionic Personality Disorder (301.50), Narcissistic Personality Disorder (301.81), Avoidant Personality Disorder (301.82), Dependent Personality Disorder (301.6), Obsessive-Compulsive Personality Disorder (301.4) and Personality Disorder Not Otherwise Specified (301.9).

The compounds of the invention may also be of use in the treatment of cognitive impairment. Within the context of the present invention, the term cognitive impairment includes for example the treatment of impairment of cognitive functions including attention, orientation, learning disorders, memory (i.e. memory disorders, amnesia, amnesic disorders, transient global amnesia syndrome and age-associated memory impairment) and language function; cognitive impairment as a result of stroke, Alzheimer's disease, Huntington's disease, Pick disease, Aids-related dementia or other dementia states such as Multiinfarct dementia, alcoholic dementia, hypotiroidism-related dementia, and dementia associated to other degenerative disorders such as cerebellar atrophy and amyotropic lateral sclerosis; other acute or sub-acute conditions that may cause cognitive decline such as delirium or depression (pseudodementia states) trauma, head trauma, age related cognitive decline, stroke, neurodegeneration, drug-induced states, neurotoxic agents, mild cognitive impairment, age related cognitive impairment, autism related cognitive impairment, Down's syndrome, cognitive deficit related to psychosis, and post-electroconvulsive treatment related cognitive disorders; and dyskinetic disorders such as Parkinson's disease, neuroleptic-induced parkinsonism, and tardive dyskinesias.

The compounds of the present invention may also be of use for the treatment of cognition impairment which arises in association or as a result of other diseases such as schizophrenia, bipolar disorder, depression, other psychiatric disorders and psychotic conditions associated with cognitive impairment.

The compounds of the invention may also be of use in the treatment of sexual dysfunctions including Sexual Desire Disorders such as Hypoactive Sexual Desire Disorder (302.71), and Sexual Aversion Disorder (302.79); sexual arousal disorders such as Female Sexual Arousal Disorder (302.72) and Male Erectile Disorder (302.72); orgasmic disorders such as Female Orgasmic Disorder (302.73), Male Orgasmic Disorder (302.74) and Premature Ejaculation (302.75); sexual pain disorder such as Dyspareunia (302.76) and Vaginismus (306.51); Sexual Dysfunction Not Otherwise Specified (302.70); paraphilias such as Exhibitionism (302.4), Fetishism (302.81), Frotteurism (302.89), Pedophilia (302.2), Sexual Masochism (302.83), Sexual Sadism (302.84), Transvestic Fetishism (302.3), Voyeurism (302.82) and Paraphilia Not Otherwise Specified (302.9); gender identity disorders such as Gender Identity Disorder in Children (302.6) and Gender Identity Disorder in Adolescents or Adults (302.85); and Sexual Disorder Not Otherwise Specified (302.9).

The compounds of the invention may also be of use as anticonvulsants. The compounds of the invention are thus useful in the treatment of convulsions in mammals, and particularly epilepsy in humans. “Epilepsy” is intended to include the following seizures: simple partial seizures, complex partial seizures, secondary generalised seizures, generalised seizures including absence seizures, myoclonic seizures, clonic seizures, tonic seizures, tonic clonic seizures and atonic seizures. The invention also provides a method of treating convulsions, which comprises administering to a mammal in need thereof an effective amount of a compound of formula (I) as hereinbefore described or a salt thereof. Treatment of epilepsy may be carried out by the administration of a non-toxic anticonvulsant effective amount of a compound of the formula (I) or a salt thereof.

The compounds of the invention may also considered to be of use in the treatment of neuropathic pain, for example in diabetic neuropathy, sciatica, non-specific lower back pain, multiple sclerosis pain, fibromyalgia, HIV-related neuropathy, neuralgia such as post-herpetic neuralgia and trigeminal neuralgia and pain resulting from physical trauma, amputation, cancer, toxins or chronic inflammatory conditions.

As used herein, the terms “treatment” and “treating” refer to the alleviation and/or cure of established symptoms as well as prophylaxis.

The invention thus provides compounds of formula (I) and salts thereof for use in therapy.

In another aspect of the present invention, there is provided a method of treating a disorder mediated by GlyT1 comprising administrating a compound of formula (I) or a salt thereof.

In order to use a compound of the present invention in therapy, it will normally be formulated into a pharmaceutical composition in accordance with standard pharmaceutical practice. The present invention also provides a pharmaceutical composition, which comprises a compound of formula (I) or a salt thereof and at least one pharmaceutically acceptable excipient.

In a further aspect, the present invention provides a process for preparing a pharmaceutical composition, the process comprising mixing a compound of formula (I) or a salt thereof and at least one pharmaceutically acceptable excipient.

A pharmaceutical composition of the invention is usually adapted for oral, sub-lingual, buccal, parenteral (for example, subcutaneous, intramuscular, or intravenous), rectal, topical and intranasal administration and in forms suitable for administration by inhalation or insufflation (either through the mouth or nose). The most suitable means of administration for a particular patient will depend on the nature and severity of the conditions being treated and on the nature of the active compound. In one embodiment, oral administration is provided.

Compositions suitable for oral administration may be provided as discrete units, such as tablets, capsules, cachets, or lozenges, each containing a predetermined amount of the active compound; as powders or granules; as solutions or suspensions in aqueous or non-aqueous liquids; or as oil-in-water or water-in-oil emulsions.

Compositions suitable for sublingual or buccal administration include lozenges comprising the active compound and, typically, a flavoured base, such as sugar and acacia or tragacanth and pastilles comprising the active compound in an inert base, such as gelatin and glycerin or sucrose and acacia.

Compositions suitable for parenteral administration typically comprise sterile aqueous solutions containing a predetermined concentration of the active compound; the solution may be isotonic with the blood of the intended recipient. Such solutions may be administered intravenously or by subcutaneous or intramuscular injection.

Compositions suitable for rectal administration may be provided as unit-dose suppositories comprising the active ingredient and one or more solid carriers forming the suppository base, for example, cocoa butter.

Compositions suitable for topical or intranasal application include ointments, creams, lotions, pastes, gels, sprays, aerosols and oils. Suitable carriers for such compositions include petroleum jelly, lanolin, polyethylene glycols, alcohols, and combinations thereof.

The compositions of the invention may be prepared by any suitable method, typically by uniformly and intimately admixing the active compound(s) with liquids or finely divided solid carriers, or both, in the required proportions and then, if necessary, shaping the resulting mixture into the desired shape.

For example, a tablet may be prepared by compressing an intimate mixture comprising a powder or granules of the active ingredient and one or more optional ingredients, such as a binder, lubricant, inert diluent, or surface active dispersing agent, or by moulding an intimate mixture of powdered active ingredient and inert liquid diluent.

Aqueous solutions for parenteral administration are typically prepared by dissolving the active compound in sufficient water to give the desired concentration and then rendering the resulting solution sterile and isotonic.

It will be appreciated that the precise dose administered will depend on the age and condition of the patient and the frequency and route of administration and will be at the ultimate discretion of the attendant physician. The compound may be administered in single or divided doses and may be administered one or more times, for example 1 to 4 times per day.

A proposed dose of the active ingredient for use according to the invention for oral, sub-lingual, parenteral, buccal, rectal, intranasal or topical administration to a human (of approximately 70 kg bodyweight) for the treatment of neurological and neuropsychiatric disorders mediated by a GlyT1 inhibitor, including schizophrenia, may be about 0.1 to about 1000 mg, for example about 0.5 mg to about 1000 mg, or about 1 mg to about 1000 mg, or about 5 mg to about 500 mg, or about 10 mg to about 100 mg of the active ingredient per unit dose, which could be administered, for example, 1 to 4 times per day.

The compounds of formula (I) and their salts thereof may also be suitable for combination with other therapeutic agents, such as typical and atypical antipsychotics. Thus, the present invention also provides:
i) a combination product of a compound of the invention with one or more further therapeutic agents such as one or more antipsychotics;
ii) a pharmaceutical composition comprising a combination product as defined in i) above and at least one carrier, diluent or excipient;
iii) the use of a combination as defined in i) above in the manufacture of a medicament for treating or preventing a disease or condition caused by a reduction or imbalance in glutamate receptor function in a mammal;
iv) a combination as defined in i) above for use in treating or preventing a disease or condition caused by a reduction or imbalance in glutamate receptor function in a mammal;
v) a kit-of-parts for use in the treatment of a psychotic disorder comprising a first dosage form comprising a compound of the invention and one or more further dosage forms each comprising a antipsychotic agent for simultaneous therapeutic administration.
vi) a combination as defined in i) above for use in therapy;
vii) a method of treatment or prevention of a disease or condition caused by a reduction or imbalance in glutamate receptor function in a mammal comprising administering an effective amount of a combination as defined in i) above;
viii) a combination as defined in i) above for treating or preventing a disease or condition caused by a reduction or imbalance in glutamate receptor function in a mammal;

The combination therapies of the invention may be administered adjunctively. By adjunctive administration is meant the coterminous or overlapping administration of each of the components in the form of separate pharmaceutical compositions or devices. This regime of therapeutic administration of two or more therapeutic agents is referred to generally by those skilled in the art and herein as adjunctive therapeutic administration; it is also known as add-on therapeutic administration. Any and all treatment regimes in which a patient receives separate but coterminous or overlapping therapeutic administration of the compounds of formula (I) or a salt thereof and at least one antipsychotic agent are within the scope of the current invention. In one embodiment of adjunctive therapeutic administration as described herein, a patient is typically stabilised on a therapeutic administration of one or more of the of the components for a period of time and then receives administration of another component. Within the scope of this invention, the compounds of formula (I) or a salt thereof may be administered as adjunctive therapeutic treatment to patients who are receiving administration of at least one antipsychotic agent, but the scope of the invention also includes the adjunctive therapeutic administration of at least one antipsychotic agent to patients who are receiving administration of compounds of formula (I) or a salt thereof.

The combination therapies of the invention may also be administered simultaneously. By simultaneous administration is meant a treatment regime wherein the individual components are administered together, either in the form of a single pharmaceutical composition or device comprising or containing both components, or as separate compositions or devices, each comprising one of the components, administered simultaneously. Such combinations of the separate individual components for simultaneous combination may be provided in the form of a kit-of-parts.

In a further aspect therefore, the invention provides a method of treatment of a psychotic disorder by adjunctive therapeutic administration of compounds of formula (I) or a salt thereof to a patient receiving therapeutic administration of at least one antipsychotic agent. In a further aspect, the invention provides the use of compounds of formula (I) or a salt thereof in the manufacture of a medicament for adjunctive therapeutic administration for the treatment of a psychotic disorder in a patient receiving therapeutic administration of at least one antipsychotic agent. The invention further provides compounds of formula (I) or a salt thereof for use for adjunctive therapeutic administration for the treatment of a psychotic disorder in a patient receiving therapeutic administration of at least one antipsychotic agent.

In a further aspect, the invention provides a method of treatment of a psychotic disorder by adjunctive therapeutic administration of at least one antipsychotic agent to a patient receiving therapeutic administration of compounds of formula (I) or a salt thereof. In a further aspect, the invention provides the use of at least one antipsychotic agent in the manufacture of a medicament for adjunctive therapeutic administration for the treatment of a psychotic disorder in a patient receiving therapeutic administration of compounds of formula (I) or a salt thereof. The invention further provides at least one antipsychotic agent for adjunctive therapeutic administration for the treatment of a psychotic disorder in a patient receiving therapeutic administration of compounds of formula (I) or a salt thereof.

In a further aspect, the invention provides a method of treatment of a psychotic disorder by simultaneous therapeutic administration of compounds of formula (I) or a salt thereof in combination with at least one antipsychotic agent. The invention further provides the use of a combination of compounds of formula (I) or a salt thereof and at least one antipsychotic agent in the manufacture of a medicament for simultaneous therapeutic administration in the treatment of a psychotic disorder. The invention further provides a combination of compounds of formula (I) or a salt thereof and at least one antipsychotic agent for simultaneous therapeutic administration in the treatment of a psychotic disorder. The invention further provides the use of compounds of formula (I) or a salt thereof in the manufacture of a medicament for simultaneous therapeutic administration with at least one antipsychotic agent in the treatment of a psychotic disorder. The invention further provides compounds of formula (I) or a salt thereof for use for simultaneous therapeutic administration with at least one antipsychotic agent in the treatment of a psychotic disorder. The invention further provides the use of at least one antipsychotic agent in the manufacture of a medicament for simultaneous therapeutic administration with compounds of formula (I) or a salt thereof in the treatment of a psychotic disorder.

In further aspects, the invention provides a method of treatment of a psychotic disorder by simultaneous therapeutic administration of a pharmaceutical composition comprising compounds of formula (I) or a salt thereof and at least one mood stabilising or antimanic agent, a pharmaceutical composition comprising compounds of formula (I) or a salt thereof and at least one mood stabilising or antimanic agent, the use of a pharmaceutical composition comprising compounds of formula (I) or a salt thereof and at least one mood stabilising or antimanic agent in the manufacture of a medicament for the treatment of a psychotic disorder, and a pharmaceutical composition comprising compounds of formula (I) or a salt thereof and at least one mood stabilising or antimanic agent for use in the treatment of a psychotic disorder.

Examples of antipsychotic drugs that are useful in the present invention include, but are not limited to: butyrophenones, such as haloperidol, pimozide, and droperidol; phenothiazines, such as chlorpromazine, thioridazine, mesoridazine, trifluoperazine, perphenazine, fluphenazine, thiflupromazine, prochlorperazine, and acetophenazine; thioxanthenes, such as thiothixene and chlorprothixene; thienobenzodiazepines; dibenzodiazepines; benzisoxazoles; dibenzothiazepines; imidazolidinones; benziso-thiazolyl-piperazines; triazine such as lamotrigine; dibenzoxazepines, such as loxapine; dihydroindolones, such as molindone; aripiprazole; and derivatives thereof that have antipsychotic activity.

Examples of tradenames and suppliers of selected antipsychotic drugs are as follows: clozapine (available under the tradename CLOZARIL®, from Mylan, Zenith Goldline, UDL, Novartis); olanzapine (available under the tradename ZYPREX®, from Lilly); ziprasidone (available under the tradename GEODON®, from Pfizer); risperidone (available under the tradename RISPERDAL®, from Janssen); quetiapine fumarate (available under the tradename SEROQUEL®, from AstraZeneca); haloperidol (available under the tradename HALDOL®, from Ortho-McNeil); chlorpromazine (available under the tradename THORAZINE®, from SmithKline Beecham (GSK)); fluphenazine (available under the tradename PROLIXIN®, from Apothecon, Copley, Schering, Teva, and American Pharmaceutical Partners, Pasadena); thiothixene (available under the tradename NAVANE®, from Pfizer); trifluoperazine (10-[3-(4-methyl-1-piperazinyl)propyl]-2-(trifluoromethyl)phenothiazine dihydrochloride, available under the tradename STELAZINE®, from Smith Klein Beckman); perphenazine (available under the tradename TRILAFON®; from Schering); thioridazine (available under the tradename MELLARIL®; from Novartis, Roxane, HiTech, Teva, and Alpharma); molindone (available under the tradename MOBAN®, from Endo); and loxapine (available under the tradename LOXITANE®; from Watson). Furthermore, benperidol (Glianimon®), perazine (Taxilan®) or melperone (Eunerpan®) may be used. Other antipsychotic drugs include promazine (available under the tradename SPARINE®), triflurpromazine (available under the tradename VESPRIN®), chlorprothixene (available under the tradename TARACTAN®), droperidol (available under the tradename INAPSINE®), acetophenazine (available under the tradename TINDAL®), prochlorperazine (available under the tradename COMPAZINE®), methotrimeprazine (available under the tradename NOZINAN®), pipotiazine (available under the tradename PIPOTRIL®), ziprasidone, and hoperidone.

It will be appreciated by those skilled in the art that the compounds according to the invention may advantageously be used in conjunction with one or more other therapeutic agents, for instance, antidepressant agents such as 5HT3 antagonists, serotonin agonists, NK-1 antagonists, selective serotonin reuptake inhibitors (SSRI), noradrenaline re-uptake inhibitors (SNRI), tricyclic antidepressants, dopaminergic antidepressants, H3 antagonists, 5HT1A antagonists, 5HT1B antagonists, 5HT1D antagonists, D1 agonists, M1 agonists and/or anticonvulsant agents, as well as cognitive enhancers.

Suitable 5HT3 antagonists which may be used in combination of the compounds of the inventions include for example ondansetron, granisetron, metoclopramide.

Suitable serotonin agonists which may be used in combination with the compounds of the invention include sumatriptan, rauwolscine, yohimbine, metoclopramide.

Suitable SSRIs which may be used in combination with the compounds of the invention include fluoxetine, citalopram, femoxetine, fluvoxamine, paroxetine, indalpine, sertraline, zimeldine.

Suitable SNRIs which may be used in combination with the compounds of the invention include venlafaxine and reboxetine.

Suitable tricyclic antidepressants which may be used in combination with a compound of the invention include imipramine, amitriptiline, chlomipramine and nortriptiline.

Suitable dopaminergic antidepressants which may be used in combination with a compound of the invention include bupropion and amineptine.

Suitable anticonvulsant agents which may be used in combination of the compounds of the invention include for example divalproex, carbamazepine and diazepam.

The invention is further illustrated by the following non-limiting examples.

The starting material may not necessarily have been prepared from the batch detailed in the relevant Description. All quoted retention times are as measured using LC/MS (Liquid Chromatography/Mass Spectrometry). Where appropriate, these retention times were used as a guide for purification using mass-directed auto-preparation (MDAP), which refers to purification by HPLC, wherein fraction collection is triggered by detection of the programmed mass ion for the compound of interest.

Where reactions are described as having been carried out in a similar manner to earlier, more completely described reactions, the general reaction conditions used were essentially the same. Work up conditions used were of the types standard in the art, but may have been adapted from one reaction to another.

Starting materials were obtained from commercial suppliers and used without further purification unless otherwise stated. Flash chromatography was carried out using pre-packed Isolute Flash™ or Biotage™ silica-gel columns as the stationary phase and analytical grade solvents as the eluent unless otherwise stated.

¹H-NMR spectra were obtained at 294K at 400 MHz frequency using either a Bruker™ DPX400 or AV400 machine and run as a dilute solution of CDCl₃ unless otherwise stated. ¹⁹F NMR was run at 294K using either a Bruker™ DPX400 or AV400 machine, at 376 MHz. All ¹H-NMR spectra were referenced to tetramethylsilane (TMS δ_{H} 0, δC 0). All coupling constants are reported in hertz (Hz), and multiplicities are labelled s (singlet), bs (broad singlet), d (doublet), t (triplet), q (quartet), dd (doublet of doublets), dt (doublet of triplets) and m (multiplet).

Total ion current traces were obtained for electrospray positive and negative ionisation (ES+/ES−) and/or atmospheric pressure chemical positive and negative ionisation (AP+/AP−).

Unless otherwise stated, all compounds with chiral centre(s) are racemic.

### ABBREVIATIONS

DCM dichloromethane
DMF dimethylformamide
HATU N-[(dimethylamino)-1H-1,2,3-triazolo[4,5-b]pyridine-1-ylmethylene]-N-methylmethanaminium hexafluorophosphate N-oxide
g grams
ml millilitres
mmol millimoles
EtOAc ethyl acetate
H—Y Zeolites refers to product CBV400 from Zeolyst, Oosterhorn, Netherlands.

### Analytical LC/MS Chromatography Conditions:

| | |
|---|---|
| Column: | Waters Atlantis 50 mm × 4.6 mm, |
| | 3 μm particle size |
| Mobile phase: | A: 0.05% Formic acid + Water |
| | B: Acetonitrile + 0.05% Formic acid |
| Gradient: | 5-min runtime: 3% B to 97% B over 4 min |
| Flow rate: | 3 ml/min |
| UV wavelength range: | 220-330 nm |
| Temperature: | 30° C. |
| Or | |
| Column: | Supelcosil ABZ + Plus 33 mm × 4.6 mm, |
| | 3 μm particle size |
| Mobile phase: | A: 10%[CH₃CN + 0.05% TFA] |
| | B: 90%[CH₃CN + 0.05% TFA] |
| Gradient: | 2.8-min runtime A:B over 2.2 min |
| Flow rate: | 0.9 ml/min |
| Temperature: | 45° C. |

### Mass Directed Auto-Purification System Chromatography Conditions:

| | |
|---|---|
| Column: | Waters Atlantis 19 mm × 100 mm or 30 mm × 100 mm, |
| | 5 μm particle size |
| Mobile phase: | A: 0.1% Formic acid + Water |
| | B: Acetonitrile + 0.1% Formic acid |
| Gradient: | 13.5 min runtime with 10 min gradient dependant on |
| | analytical retention time |
| Flow rate: | 20 or 40 ml/min |

### General:

Where reactions are described as having been carried out in a similar manner to earlier, more completely described reactions, the general reaction conditions used were essentially the same. Work up conditions used were of the types standard in the art, but may have been adapted from one reaction to another.

In the procedures that follow, reference to an Intermediate or Example by number is typically provided. This is provided merely for assistance to the skilled chemist to identify the starting material used.

### Description 1: Methyl amino(4-chlorophenyl)acetate

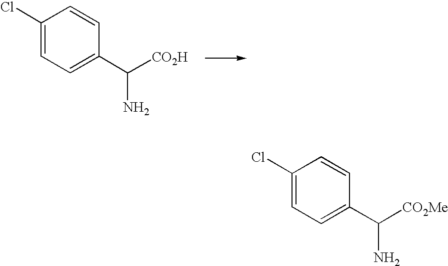

To ice-chilled methanol (300 ml) under argon was carefully added dropwise thionyl chloride (15.44 ml; 0.217 mol) over 45 min. 4-Chlorophenylglycine (26.26 g; 0.141 mol) was added, ice cooling removed and the reaction mixture warmed to 40° C. The reaction was stirred at 40° C. for 48 h. After cooling to room temperature, the reaction was evaporated under reduced pressure. Re-evaporation from methanol afforded a white solid which was triturated with diethyl ether (ca. 700 ml) and then stored at ca. 4° C. for 64 h, filtered, washed with diethyl ether and dried in vacuo to afford the title product as the hydrochloride salt (33.40 g; 100%). ¹H NMR (d₆-DMSO) δ: 3.72 (3H, s), 5.36 (1H, s), 7.53-7.58 (4H, m), 9.07 (3H, s). Mass Spectrum (Electrospray LC/MS): Found 200 (MH⁺). C₉H₁₀³⁵ClNO₂ requires 199. Ret. time 1.32 min.

### Description 2: 2-Amino-2-(4-chlorophenyl)acetamide

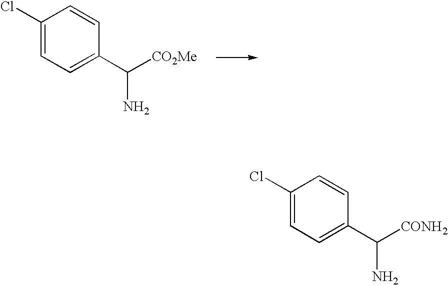

Methyl amino(4-chlorophenyl)acetate D1 as the hydrochloride salt (33.40 g; 0.14 mol) was dissolved in 0.88 ammonia (500 ml; ca. 7.4 mol) and stirred at room temperature for 64 h. The reaction mixture was extracted with DCM (300 ml×6), the extracts dried (Na₂SO₄) and evaporated under reduced pressure to a white solid, which was dried under reduced pressure to afford the title product (22.45 g; 86%). ¹H NMR (CDCl₃) δ: 1.82 (2H, br s), 4.53 (1H, s), 5.49 (1H, br s), 6.92 (1H, br s), 7.32-7.39 (4H, m).

### Description 3: 3-(4-Chlorophenyl)-8-oxa-1,4-diazaspiro[4.5]decan-2-one

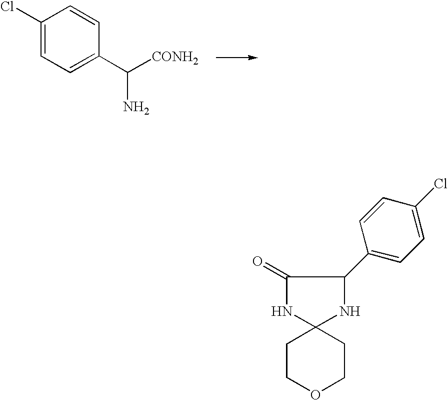

A mixture of 2-amino-2-(4-chlorophenyl)acetamide D2 (800 mg; 4.33 mmol) and tetrahydro-4H-pyran-4-one (433 mg; 4.33 mmol) in methanol (40 ml) containing H—Y zeolites (2 g) was heated at reflux for 15 h. The reaction mixture was filtered through Kieselguhr and the filtrate evaporated under reduced pressure to afford the title product (897 mg; 78%) as a white solid which was used without further purification. ¹H NMR (CDCl₃) inter alia 6: 1.74-1.94 (4H, m), 2.28 (1H, s), 3.7-3.9 (4H, m), 4.70 (1H, s), 6.89 (1H, s), 7.33-7.36 (2H, m), 7.46-7.48 (2H, m).

### Description 4: 3-(4-chlorophenyl)-8-oxa-1,4-diazaspiro[4.5]dec-3-en-2-one

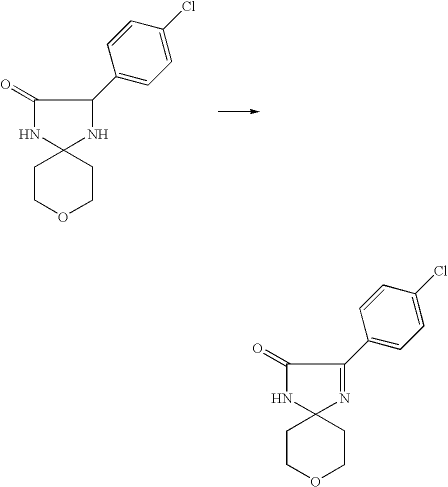

A mixture of 3-(4-chlorophenyl)-8-oxa-1,4-diazaspiro[4.5]decan-2-one D3 (897 mg; 3.36 mmol) and N-bromosuccinimide (600 mg; 3.36 mmol) in DCM (40 ml) was stirred at room temperature overnight. Saturated aqueous sodium bicarbonate was added and the organic layers separated and dried through a phase-separation cartridge. Evaporation under reduced pressure and chromatography of the residue on silica gel eluting with an ethyl acetate-pentane gradient afforded the title product (600 mg; 67%) as a white solid. ¹H NMR (CDCl₃) δ: 1.87-1.94 (4H, m), 3.87-3.93 (2H, m), 4.09-4.15 (2H, m), 7.44-7.47 (2H, m), 8.39-8.43 (2H, m), 8.59 (1H, s). Mass Spectrum (Electrospray LC/MS): Found 263 (MH⁺). C₁₃H₁₃³⁵ClN₂O₂ requires 262. Ret. time 2.43 min.

### Description 5: Ethyl[3-(4-chlorophenyl)-2-oxo-8-oxa-1,4-diazaspiro[4.5]dec-3-en-1-yl]acetate

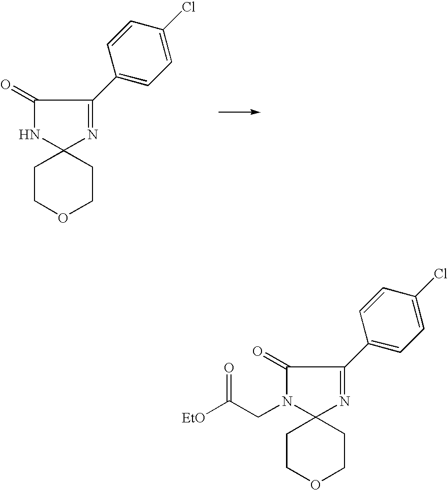

A mixture of 3-(4-chlorophenyl)-8-oxa-1,4-diazaspiro[4.5]dec-3-en-2-one D4 (300 mg; 1.13 mmol) and ethyl bromoacetate (0.13 ml; 1.2 mmol) in acetone (30 ml) was stirred with potassium carbonate (166 mg; 1.2 mmol) at room temperature overnight and then heated at reflux for 2 h. Further ethyl bromoacetate (0.26 ml; 2.4 mmol) was added and heating continued for ca. 72 h. Acetone was removed under reduced pressure and the residue partitioned between water and DCM. The organic layers were separated and dried using a phase-separation cartridge and evaporated under reduced pressure to afford the title product (450 mg), which was used without further purification. Mass Spectrum (Electrospray LC/MS): Found 351 (MH⁺). C₁₇H₁₉³⁵ClN₂O₄ requires 350. Ret. time 3.11 min.

### Description 6: [3-(4-Chlorophenyl)-2-oxo-8-oxa-1,4-diazaspiro[4.5]dec-3-en-1-yl]acetic acid

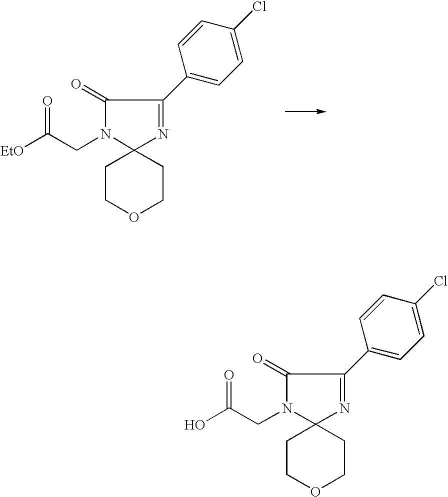

A mixture of ethyl [3-(4-chlorophenyl)-2-oxo-8-oxa-1,4-diazaspiro[4.5]dec-3-en-1-yl]acetate D5 (450 mg; 1.28 mmol) and sodium hydroxide (150 mg; 3.75 mmol) in ethanol (30 ml) and water (30 ml) was heated at reflux overnight. The ethanol and water were evaporated under reduced pressure and the white residue acidified with 2M hydrochloric acid. Extraction with ethyl acetate (×2) and drying the combined extracts with a phase-separation cartridge containing sodium sulphate and evaporation afforded the title product (353 mg; 85%) as a white solid. Mass Spectrum (Electrospray LC/MS): Found 323 (MH⁺). C₁₅H₁₅³⁵ClN₂O₄ requires 322. Ret. time 2.49 min.

### Description 7: 2-Chloro-N-(3,4-difluorophenyl)acetamide

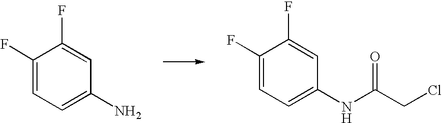

A mixture of 3,4-difluoroaniline (6.46 g; 50 mmol) and chloroacetyl chloride (5.65 g; 50 mmol) in dioxan (50 ml) was heated with stirring for 1 h. The solution was concentrated, cooled to room temperature and water added. The resulting precipitate was filtered off and dried to afford the title product (9.41 g; 91.5%). ¹H NMR (CDCl₃) δ: 4.20 (2H, s), 7.13-7.16 (2H, m), 7.63-7.68 (1H, s), 8.23 (1H, brs).

### Description 8: 1,1-Dimethylethyl 2-(4-chlorophenyl)-3-oxo-1,4,8-triazaspiro[4.5]decane-8-carboxylate

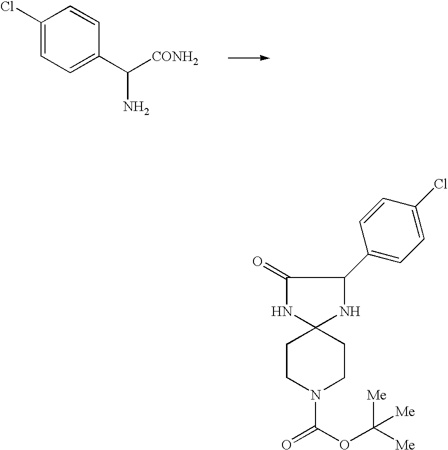

A mixture of 2-amino-2-(4-chlorophenyl)acetamide D2 (1.0 g; 5.4 mmol) and 1,1-dimethylethyl 4-oxo-1-piperidinecarboxylate (1.08 g; 5.4 mmol) in ethanol was heated at reflux for 20 h. Evaporation afforded the title product (ca. 2 g) which was used without further purification.

### Description 9: 1,1-Dimethylethyl 2-(4-chlorophenyl)-3-oxo-1,4,8-triazaspiro[4.5]dec-1-ene-8-carboxylate

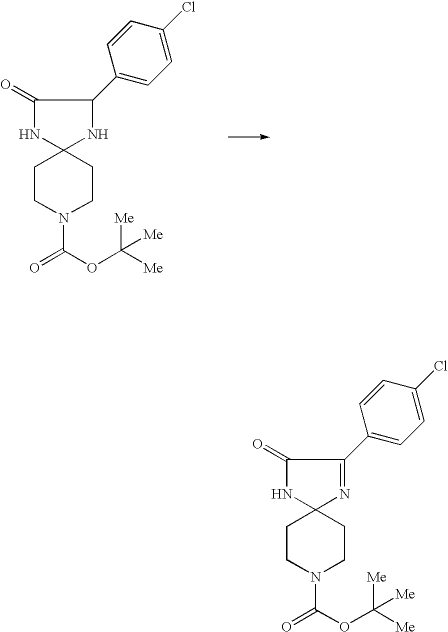

The title compound (1.2 g; 63%) was obtained from 1,1-dimethylethyl 2-(4-chlorophenyl)-3-oxo-1,4,8-triazaspiro[4.5]decane-8-carboxylate D8 (1.9 g; 5.2 mmol) and N-bromosuccinimide (0.925 g; 5.2 mmol) using a similar method to that described in D4. ¹H NMR (CDCl₃) δ: 1.50 (9H, s), 1.76-1.88 (4H, m), 3.66-3.77 (4H, m), 7.43-7.46 (2H, m), 7.72 (1H, s), 8.37-8.41 (2H, m).

### Description 10:1,1-Dimethylethyl 2-(4-chlorophenyl)-4-{2-[(3,4-difluorophenyl)amino]-2-oxoethyl}-3-oxo-1,4,8-triazaspiro[4.5]dec-1-ene-8-carboxylate

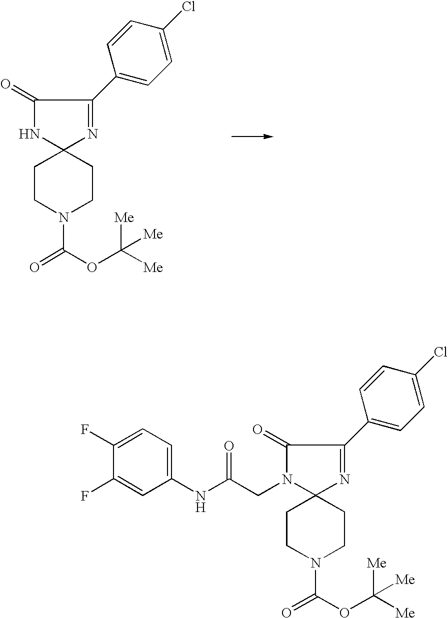

To a solution of 1,1-dimethylethyl 2-(4-chlorophenyl)-3-oxo-1,4,8-triazaspiro[4.5]dec-1-ene-8-carboxylate D9 (500 mg; 1.37 mmol) in DMF (30 ml) was added sodium hydride (55 mg; 60% dispersion in oil; 1.37 mmol) at room temperature, followed by 2-chloro-N-(3,4-difluorophenyl)acetamide D7 (300 mg; 1.46 mmol). After stirring overnight the reaction mixture was partitioned between ethyl acetate and water, the organic layers separated and further washed with water (×2). The organic was dried through a phase separation cartridge, evaporated and the residue chromatographed on silica, eluting with an ethyl acetate—pentane gradient to afford the title compound (125 mg; 16%). ¹H NMR (CDCl₃) δ: 1.34-1.37 (2H, m), 1.50 (9H, s), 2.17-2.25 (2H, m), 3.39 (2H, br s), 4.10-4.31 (4H, br s), 7.03-7.07 (2H, m), 7.46-7.49 (2H, m), 7.54-7.59 (1H, s), 8.41-8.45 (2H, m), 8.90 (1H, s). Mass Spectrum (Electrospray LC/MS): Found 477 (MH⁺-tBu). C₂₆H₂₇³⁵ClF₂N₄O₄ requires 532. Ret. time 3.69 min.

### Description 11: 2-[3-(4-Chlorophenyl)-2-oxo-1,4,8-triazaspiro[4.5]dec-3-en-1-yl]-N-(3,4-difluorophenyl)acetamide

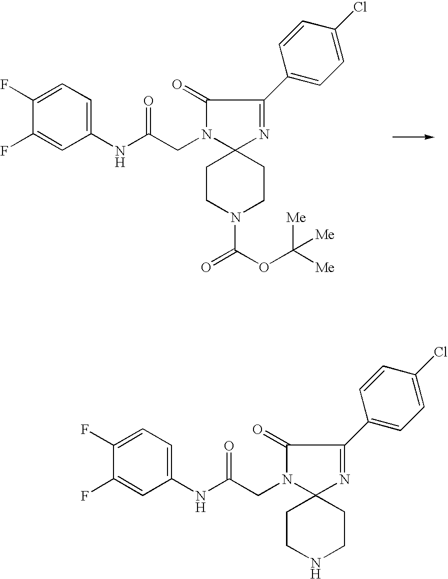

To 1,1-dimethylethyl 2-(4-chlorophenyl)-4-{2-[(3,4-difluorophenyl)amino]-2-oxoethyl}-3-oxo-1,4,8-triazaspiro[4.5]dec-1-ene-8-carboxylate D11 (110 mg; 0.21 mmol) was added 4M hydrogen chloride in dioxan (1 ml; 4 mmol). After 2 h the solvent was removed with a stream of argon and the residue dried in a vacuum pistol at 40° C. overnight to afford the title product (109 mg; 100%) as a hydrochloride salt. Mass Spectrum (Electrospray LC/MS): Found 433 (MH⁺). C₂₁H₁₉³⁵ClF₂N₄O₂ requires 432. Ret. time 2.06 min.

### Description 12: 1,1-Dimethylethyl 2-(4-chlorophenyl)-3-oxo-1,4,7-triazaspiro[4.5]decane-7-carboxylate

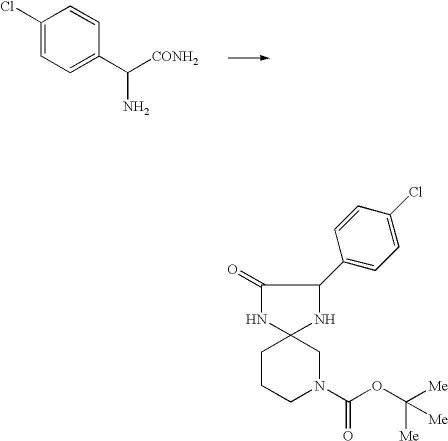

The title product (2.9 g) was obtained from 2-amino-2-(4-chlorophenyl)acetamide D2 (1.5 g; 8 mmol) and 1,1-dimethylethyl 3-oxo-1-piperidinecarboxylate (1.62 g; 8 mmol) using a similar method to that described in D8 and was used without purification.

### Description 13: 1,1-Dimethylethyl 2-(4-chlorophenyl)-3-oxo-1,4,7-triazaspiro[4.5]dec-1-ene-7-carboxylate

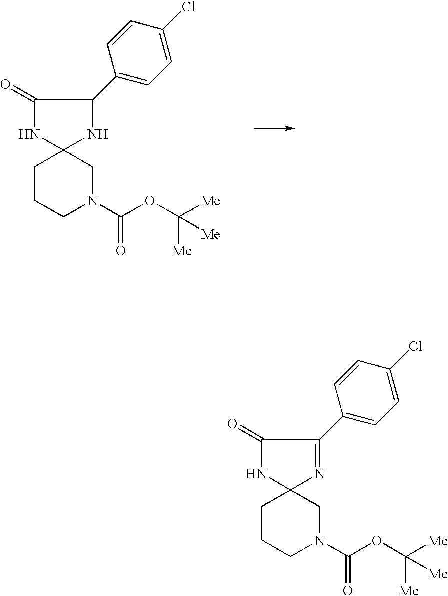

The title compound (3.12 g) was obtained from 1,1-dimethylethyl 2-(4-chlorophenyl)-3-oxo-1,4,7-triazaspiro[4.5]decane-7-carboxylate D12 (2.9 g; 8 mmol) and N-bromosuccinimide (1.42 g; 8 mmol) using a similar method to that described in D4. Mass Spectrum (Electrospray LC/MS): Found 364 (MH⁺). C₁₈H₂₂³⁵ClN₃O₃ requires 363. Ret. time 3.08 min.

### Description 14: 1,1-Dimethylethyl 2-(4-chlorophenyl)-4-{2-[(3,4-difluorophenyl)amino]-2-oxoethyl}-3-oxo-1,4,7-triazaspiro[4.5]dec-1-ene-7-carboxylate

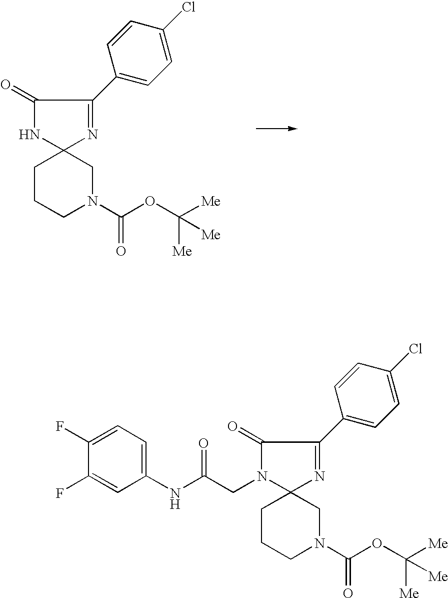

A mixture of 1,1-dimethylethyl 2-(4-chlorophenyl)-3-oxo-1,4,7-triazaspiro[4.5]dec-1-ene-7-carboxylate D13 (363 mg; μmol), 2-chloro-N-(3,4-difluorophenyl)acetamide D7 (238 mg; 1.2 mmol) and potassium carbonate (326 mg; 2.4 mmol) in DMF (10 ml) was heated at 60° C. overnight. Work-up and purification using methods similar to that described in D10 afforded the title product (240 mg; 45%). Mass Spectrum (Electrospray LC/MS): Found 533 (MH⁺). C₂₆H₂₇³⁵ClF₂N₄O₄ requires 532. Ret. time 3.60 min.

### Description 15: 2-[3-(4-Chlorophenyl)-2-oxo-1,4,7-triazaspiro[4.5]dec-3-en-1-yl]-N-(3,4-difluorophenyl)acetamide

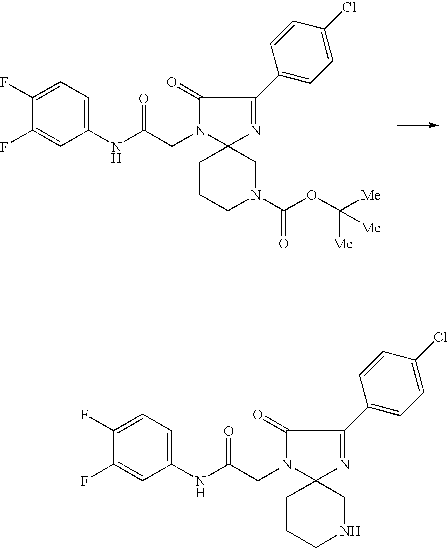

To 1,1-dimethylethyl 2-(4-chlorophenyl)-4-{2-[(3,4-difluorophenyl)amino]-2-oxoethyl}-3-oxo-1,4,7-triazaspiro[4.5]dec-1-ene-7-carboxylate D14 (240 mg; 0.45 mmol) was added 4M hydrogen chloride in dioxan (2.3 ml). After 2 h the solvent was evaporated and the residue converted to the crude free base by partition between ethyl acetate and saturated sodium bicarbonate. Chromatography on silica gel eluting with a DCM—methanol gradient afforded the title product (120 mg; 62%). Mass Spectrum (Electrospray LC/MS): Found 433 (MH⁺). C₂₁H₁₉³⁵ClF₂N₄O₂ requires 432. Ret. time 2.05 min.

### Description 16. 2-Bromo-N-[3-(trifluoromethyl)phenyl]acetamide

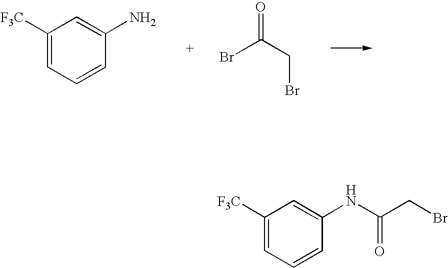

A stirred solution of 3-(trifluoromethyl)aniline (2.0 g, 0.012 mol) in dichloromethane (60 ml) at 10° C. under argon was treated dropwise over 5 minutes with bromoacetyl bromide (1.2 ml, 0.0137 mol). A white precipitate formed. This was allowed to warm to room temperature with good stirring over 1.5 hours, then treated with solid sodium hydrogen carbonate (1.65 g, 0.0196 mol) and stirred well for 40 minutes. The mixture was treated with water (100 ml), stirred well for 10 minutes, then the dichloromethane layer was isolated by passage through a phase separation cartridge and concentrated under vacuum to afford the title compound as a colourless oil (3.65 g, 100%). Mass Spectrum (Electrospray LC/MS): Found 282 (MH⁺). C₉H₇⁷⁹BrF₃NO requires 281. Ret. time 2.74 min. ¹H NMR δ (CDCl₃; 400 MHz): 4.05 (2H, s), 7.40-7.53 (2H, m), 7.76 (1H, d), 7.83 (1H, s), 8.24 (1H, br s).

### Description 17: 2-Amino-2-(4-bromophenyl)acetamide

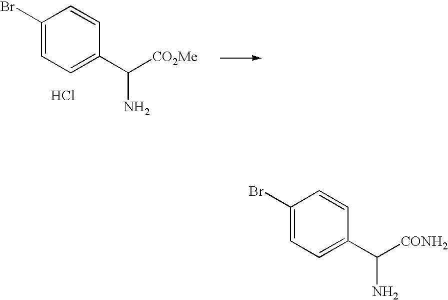

Methyl amino(4-bromophenyl)acetate hydrochloride (10 g, 41.0 mmol) in 0.88 ammonia (120 ml), was stirred at room temperature overnight. The white precipitate was collected by filtration and dried to give the title compound (4.95 g).

¹H NMR (CD₃OD) δ: 4.40 (1H, s), 4.88 (4H, m), 7.3 (2H, m), 7.5 (2H, m).

### Description 18: 3-(4-Bromophenyl)-7-oxa-1,4-diazaspiro[4.5]decan-2-one

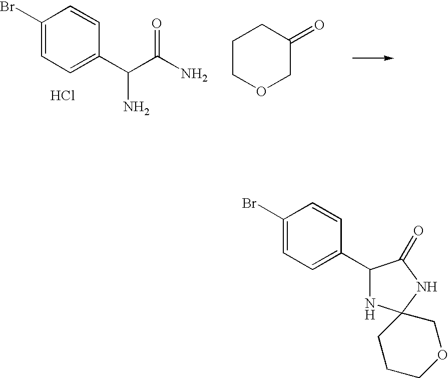

A mixture of tetrahydropyran-3-one (0.25 g) and 2-amino-2-(4-bromophenyl)acetamide (D17) (0.572 g) and H—Y Zeolites (0.57 g) in methanol (25 ml) was refluxed under argon overnight. The mixture was filtered through Kieselguhr and the solvent was removed to give crude title compound as a yellow solid which was used without purification (712 mg).

Mass spectrum Found 311/3 (MH⁺).

### Description 19: 3-(4-Bromophenyl)-7-oxa-1,4-diazaspiro[4.5]dec-3-en-2-one

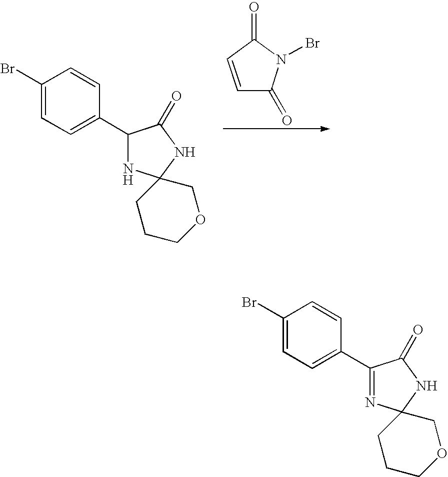

3-(4-Bromophenyl)-7-oxa-1,4-diazaspiro[4.5]decan-2-one (D18) (0.771 g) in DCM (50 ml) was treated with N-bromosuccinimide (0.463 g) then stirred under argon overnight. Sodium bicarbonate solution was added and the mixture was stirred for 1 hour then separated. The DCM layer was washed with brine, and dried over sodium sulphate and the solvent was removed to give a fawn solid. This was triturated with DCM and the white product was collected by filtration to give the title compound. (209 mg).

¹H NMR (d⁶DMSO) δ: 1.65 (1H, m), 1.85 (2H, m), 2.08 (1H, m), 3.45 (1H, m), 3.6-3.75 (2H, m), 3.8 (1H, m), 7.72 (2H, m), 8.28 (2H, m), 10.45 (1H, s).

### Description 20: 3-(4-Bromophenyl)-7-oxa-1,4-diazaspiro[4.4]nonan-2-one

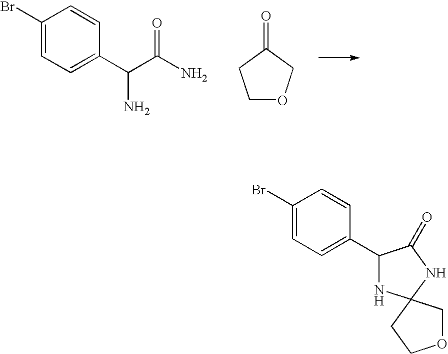

A mixture of dihydro-3(2H)-furanone (0.951 g) and 2-amino-2-(4-bromophenyl)acetamide (D17) (2.3 g) and H—Y Zeolites (2.3 g) in methanol (125 ml) was refluxed under argon overnight. The mixture was filtered through Kieselgugr and the solvent was removed to give the title compound as an off white solid foam (2.59 g) which was used without further purification.

Mass spectrum Found 297/9 (MH⁺).

### Description 21: 3-(4-Bromophenyl)-7-oxa-1,4-diazaspiro[4.4]non-3-en-2-one

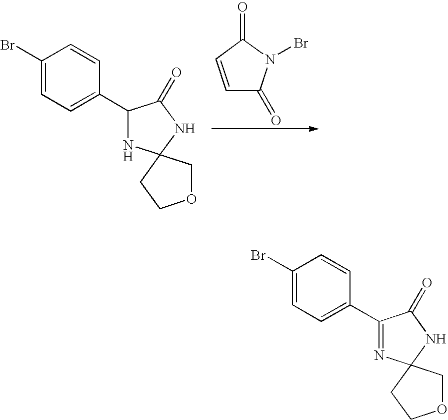

3-(4-Bromophenyl)-7-oxa-1,4-diazaspiro[4.4]nonan-2-one (D20) (2.52 g) in DCM (50 ml) was treated with N-bromosuccinimide (1.64 g) then stirred under argon overnight. Sodium bicarbonate solution was added and the mixture was stirred for 1 hour then separated. The DCM layer was washed with brine, dried sodium sulphate and the solvent was removed to give a fawn solid, this was chromatographed, on a silica column eluted with 0-5% MeOH-DCM to give the title compound as a brown solid (1.22 g).

¹H NMR (CDCl₃) δ: 2.26 (1H, m), 2.60 (1H, m), 3.85 (1H, m), 4.06 (1H, m), 4.25 (2H, m), 7.62 (2H, m), 7.86 (1H, br), 8.32 (2H, m). Mass spectrum Found 295/7 (MH⁺).

### EXAMPLE 1

### 2-[3-(4-Chlorophenyl)-2-oxo-8-oxa-1,4-diazaspiro[4.5]dec-3-en-1-yl]-N-(3,4-difluorophenyl)acetamide

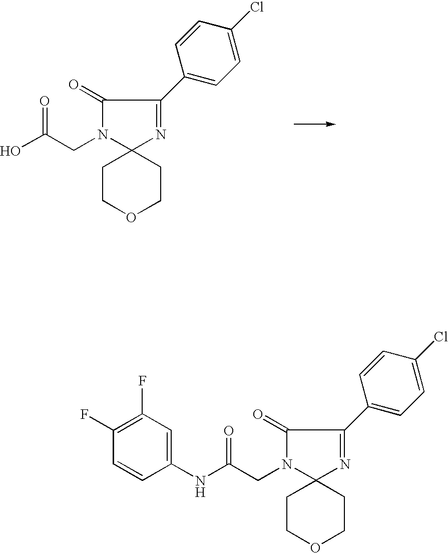

A mixture of [3-(4-chlorophenyl)-2-oxo-8-oxa-1,4-diazaspiro[4.5]dec-3-en-1-yl]acetic acid D6 (108 mg; 0.33 mmol), 3,4-difluoroaniline (47 mg; 0.36 mmol) and HATU (137 mg; 0.36 mmol) in DCM (4 ml) was shaken overnight. Further HATU (137 mg; 0.36 mmol) was added and shaking continued overnight. Saturated aqueous sodium bicarbonate was added and the organic layers separated through a phase-separation cartridge. Chromatography on silica gel eluting with an ethyl acetate-pentane gradient afforded the title product (46 mg; 32%). ¹H NMR (CDCl₃) δ: 1.30 (2H, m), 2.36-2.43 (2H, m), 4.04-4.15 (4H, m), 4.25 (2H, s), 7.04-7.10 (2H, m), 7.44-7.50 (2H, m), 7.55-7.60 (1H, s), 8.43-8.46 (2H, m), 8.86 (1H, s). Mass Spectrum (Electrospray LC/MS): Found 434 (MH⁺). C₂₁H₁₈³⁵ClF₂N₃O₃ requires 433. Ret. time 3.23 min.

The compounds in Table 1 were prepared using similar methods to those described for Example 1.

TABLE 1

| | | Mass spectrum | |
|---|---|---|---|
| | | (Electrospray | |
| | | LC/MS), API⁺ | |
| Ex | Structure | Ret. time (min) | Name |
| 2 | 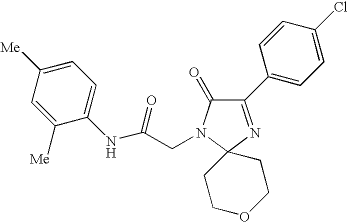 | Found 426 (MH⁺) C₂₃H₂₄³⁵ClN₃O₃ requires 425; 3.23. | 2-[3-(4-Chlorophenyl)-2- oxo-8-oxa-1,4- diazaspiro[4.5]dec-3-en- 1-yl]-N-(2,4- dimethylphenyl) acetamide |

### EXAMPLE 3

### 2-[3-(4-Chlorophenyl)-8-methyl-2-oxo-1,4,8-triazaspiro[4.5]dec-3-en-1-yl]-N-(3,4-difluorophenyl)acetamide

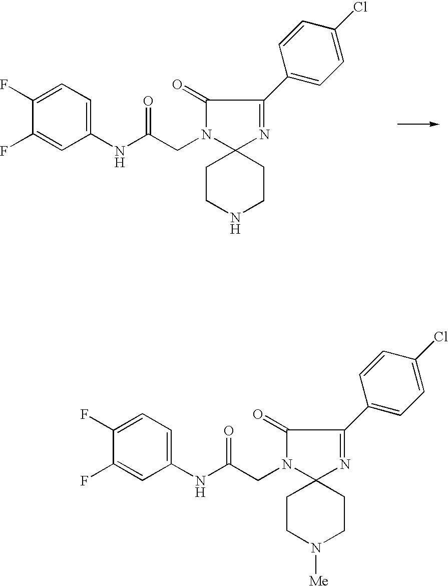

A mixture of 2-[3-(4-chlorophenyl)-2-oxo-1,4,8-triazaspiro[4.5]dec-3-en-1-yl]-N-(3,4-difluorophenyl)acetamide D11 (83 mg; 0.18 mmol), aqueous formaldehyde (0.1 ml; 37% solution in water; 1.2 mmol) and sodium triacetoxyborohydride (114 mg; 0.54 mmol) in methanol (10 ml) was stirred overnight at room temperature. The methanol was evaporated and the residue partitioned between saturated sodium bicarbonate and DCM; the organic layers were separated and dried using a phase-separation cartridge and evaporated to a white solid. ¹H NMR (CDCl₃) δ: 1.34-1.43 (2H, m), 2.35-2.47 (2H, m), 2.43 (3H, s), 2.67-2.77 (2H, m), 2.87-2.92 (2H, m), 4.24 (2H, s), 7.02-7.07 (2H, m), 7.42-7.50 (2H, m), 7.53-7.61 (1H, m), 8.41-8.46 (2H, m), 8.87 (1H, s).

The compounds in Table 2 were prepared using similar methods to those described for Example 3.

TABLE 2

| | | Mass spectrum | |
|---|---|---|---|
| | | (Electrospray | |
| | | LC/MS), API⁺ | |
| Ex | Structure | Ret. time (min) | Name |
| 4 | 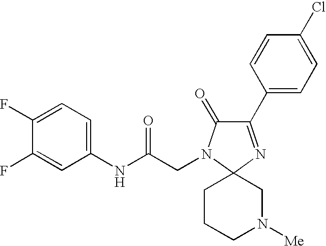 | Found 447 (MH⁺) C₂₂H₂₁³⁵ClF₂N₄O₂ requires 447; 2.04. | 2-[3-(4-chlorophenyl)-7- methyl-2-oxo-1,4,7- triazaspiro[4.5]dec-3-en- 1-yl]-N-(3,4- difluorophenyl)acetamide |

### EXAMPLE 5

### 2-[3-(4-Bromophenyl)-2-oxo-7-oxa-1,4-diazaspiro[4.5]dec-3-en-1-yl]-N-[3-(trifluoromethyl)phenyl]acetamide

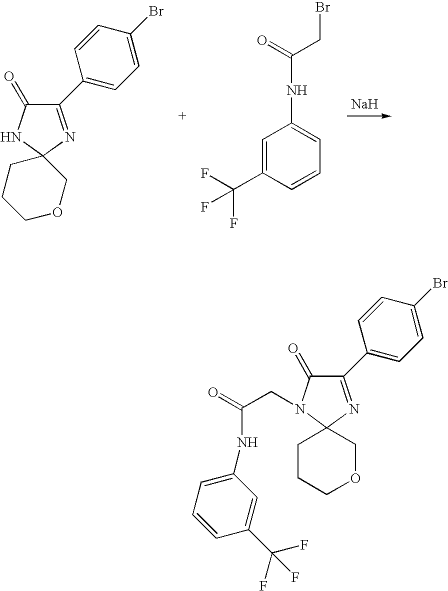

3-(4-Bromophenyl)-7-oxa-1,4-diazaspiro[4.5]dec-3-en-2-one (D19) (100 mg, 0.32 mmol) in DMF (4 ml) was cooled to ice bath temp and treated with sodium hydride (14.23 mg, 0.356 mmol) under an atmosphere of argon. The mixture was stirred for 30 minutes then 2-bromo-N-[3-(trifluoromethyl)phenyl]acetamide (D16) (100 mg, 0.356 mmol) in DMF (2 ml) was added over 1.5 hour by syringe pump. The mixture was then allowed to warm to room temp overnight. The mixture was poured into water and extracted with ethyl acetate. The ethyl acetate layer was washed with brine and dried using sodium sulphate and the solvent was removed. The residue was chromatographed on a silica column eluted with a gradient of 0-5% methanol in DCM. A mixture was obtained which was purified by low pH MDAP to give the title compound (81 mg).

¹H NMR (d⁶DMSO) δ: 1.65-1.80 (2H, m), 2.06-2.28 (2H, m), 3.5 (1H, m), 3.65 (1H, m), 3.8-3.95 (2H, m), 4.5 (2H, m), 7.42 (1H, m), 7.61 (1H, m), 7.77 (3H, m) 8.08 (1H, m), 8.32 (2H, s), 10.6 (1H, s).

¹⁹F NMR (DMSO) δ: −61.4 (s),

Mass Spectrum (Electrospray LC/MS): Found 510 (MH⁺). Ret. time 3.33 min.

### EXAMPLE 6

### 2-[3-(4-Bromophenyl)-2-oxo-7-oxa-1,4-diazaspiro[4.4]non-3-en-1-yl]-N-[3-(trifluoromethyl)phenyl]acetamide

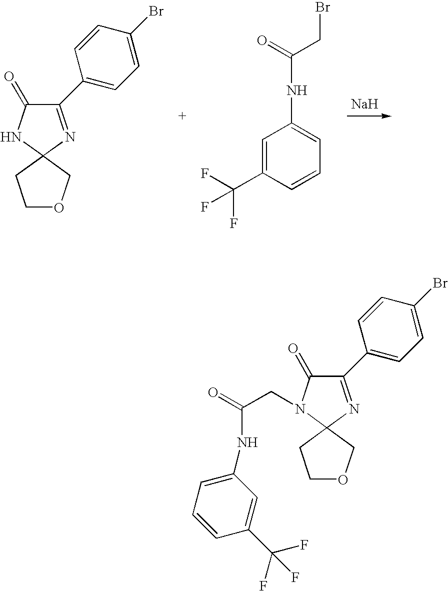

3-(4-Bromophenyl)-7-oxa-1,4-diazaspiro[4.4]non-3-en-2-one (D21) (0.6 g) in DMF (4 ml) was cooled to ice bath temp and treated with sodium hydride (81 mg) under argon. The mixture was stirred for 30 minutes when 2-bromo-N-[3-(trifluoromethyl)phenyl]acetamide (D16) (631 mg) in DMF (3 ml) was added over 2 hours by syringe pump. The mixture was then allowed to warm to room temp overnight. The solvent was partially removed then in vacuo and then poured into water and extracted with ethyl acetate. The ethyl acetate layer was dried with brine and sodium sulphate and the solvent was removed. The mixture was purified by MDAP and then crystallised by stirring with ether to give the title compound. (414 mg)

¹H NMR (CDCl₃) δ: 2.4-2.6 (2H, m), 3.96 (1H, m), 4.06 (1H, m), 4.22 (1H, m), 4.35 (3H, m), 7.4 (2H, m), 7.65 (3H, m), 7.88 (1H, m), 8.33 (2H, m) 8.75 (1H, br)

¹⁹F NMR (DMSO) δ: −62.8 (s),

Mass Spectrum (Electrospray LC/MS): Found 496/8 (MH⁺). Ret. time 3.08 min.

1-26. (canceled)

27. A compound of formula (I) or a salt thereof: 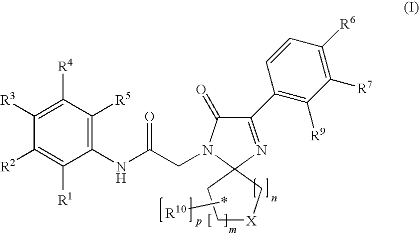
wherein:
R¹ is selected from the group consisting of H, C₁-C₄alkyl, C₁-C₄alkoxy, halo, haloC₁-C₄alkyl, haloC₁-C₄alkoxy, C₁-C₄alkylthio, C₃-C₆cycloalkyl, C₃-C₆cycloalkylC₁-C₄alkyl, C₁-C₄alkylsulfonyl, C₁-C₄alkoxyC₁-C₄alkyl, CONR^{a}R^{b} wherein R^{a} and R^{b} are independently selected from the group consisting of H and C₁-C₄alkyl; or R^{a} and R^{b}, together with the nitrogen atom to which they are attached, form a 4- to 7-membered ring, and cyano;
R² is selected from the group consisting of H, C₁-C₄alkyl, C₁-C₄alkoxy, halo, haloC₁-C₄alkyl, haloC₁-C₄alkoxy, C₁-C₄alkylthio, C₃-C₆cycloalkyl, C₃-C₆cycloalkylC₁-C₄alkyl, C₁-C₄alkylsulfonyl, C₁-C₄alkoxyC₁-C₄alkyl, CONR^{c}R^{d} wherein R^{c} and R^{d} are independently selected from H and C₁-C₄alkyl; or R^{c} and R^{d}, together with the nitrogen atom to which they are attached, form a 4- to 7-membered ring; and cyano;
R³ is selected from the group consisting of H, C₁-C₄alkyl, C₁-C₄alkoxy, halo, haloC₁-C₄alkyl, haloC₁-C₄alkoxy, C₁-C₄alkylthio, C₃-C₆cycloalkyl, C₃-C₆cycloalkylC₁-C₄alkyl, C₁-C₄alkylsulfonyl, C₁-C₄alkoxyC₁-C₄alkyl, CONR^{e}R^{f} wherein R^{e} and R^{f} are independently selected from the group consisting of H and C₁-C₄alkyl, or R^{e} and R^{f}, together with the nitrogen atom to which they are attached, form a 4- to 7-membered ring; and cyano;
or R² and R³ together form a group selected from —O—CH₂—O— and —O—CH₂—CH₂—O—;
R⁴ is selected from the group consisting of H, C₁-C₄alkyl, C₁-C₄alkoxy, halo, haloC₁-C₄alkyl, haloC₁-C₄alkoxy, C₁-C₄alkylthio, C₃-C₆cycloalkyl, C₃-C₆cycloalkylC₁-C₄alkyl, C₁-C₄alkylsulfonyl, C₁-C₄alkoxyC₁-C₄alkyl, CONR^{g}R^{h} wherein R^{g} and R^{h} are independently selected from the group consisting of H and C₁-C₄alkyl, or R^{g} and R^{h}, together with the nitrogen atom to which they are attached, form a 4- to 7-membered ring) and cyano;
R⁵ is selected from the group consisting of hydrogen, chloro, fluoro, C₁-C₄alkyl and CF₃;
R⁶ is selected from the group consisting of H, C₁-C₄alkyl, C₁-C₄alkoxy, haloC₁-C₄alkyl, haloC₁-C₄alkoxy, halo, cyano, C₁-C₄alkoxyC₁-C₄alkyl and C₁-C₄alkoxyC₁-C₄alkoxy;
R⁷ is selected from the group consisting of H, C₁-C₄alkyl, C₁-C₄alkoxy, haloC₁-C₄alkyl, haloC₁-C₄alkoxy, halo, cyano, C₁₋₄alkoxyC₁₋₄alkyl and C₁-C₄alkoxyC₁-C₄alkoxy;
X is O or NR⁸;
R⁸ is selected from the group consisting of H and C₁-C₃alkyl;
m is selected from the group consisting of 0, 1, 2 and 3, and n is selected from the group consisting of 1, 2 and 3, wherein m+n is 1, 2, 3, 4 or 5;
R⁹ is selected from the group consisting of H and fluoro;
R¹⁰ is independently selected from the group consisting of H and C₁-C₄alkyl; and
p is selected from the group consisting of 1, 2, 3 and 4.

28. A compound as claimed in claim 27 wherein R¹ is selected from H and methyl.

29. A compound as claimed in claim 27 wherein R² is selected from H, halo and halomethyl.

30. A compound as claimed in 27 wherein R² is selected from the group consisting of H, fluoro and trifluoromethyl.

31. A compound as claimed in claim 27 wherein R³ is selected from the group consisting of H, methyl, chloro and fluoro.

32. A compound as claimed in claim 27 wherein R⁴ is selected from the group consisting of H, fluoro and trifluoromethyl.

33. A compound as claimed in claim 27 wherein R⁵ is H.

34. A compound as claimed in claim 27 wherein R⁶ is chloro or bromo.

35. A compound as claimed in claim 27 wherein R⁷ is H.

36. A compound as claimed in claim 27 wherein X is O.

37. A compound as claimed in claim 27 wherein X is NR⁸ and R⁸ is methyl.

38. A compound as claimed in claim 27 wherein R⁹ is H.

39. A compound as claimed in claim 27 wherein R¹⁰ is H.

40. A compound as claimed in claim 27 wherein n is selected from the group consisting of 1 and 2.

41. A compound as claimed in claim 27 wherein m is selected from the group consisting of 1 and 2.

42. A compound as claimed in claim 27 wherein p is selected from the group consisting of 1 and 2.

43. A compound as claimed in claim 27 which is selected from the group consisting of:
2-[3-(4-Chlorophenyl)-2-oxo-8-oxa-1,4-diazaspiro[4.5]dec-3-en-1-yl]-N-(3,4-difluorophenyl)acetamide;
2-[3-(4-Chlorophenyl)-2-oxo-8-oxa-1,4-diazaspiro[4.5]dec-3-en-1-yl]-N-(2,4-dimethylphenyl)acetamide;
2-[3-(4-Chlorophenyl)-8-methyl-2-oxo-1,4,8-triazaspiro[4.5]dec-3-en-1-yl]-N-(3,4-difluorophenyl)acetamide;
2-[3-(4-Chlorophenyl)-7-methyl-2-oxo-1,4,7-triazaspiro[4.5]dec-3-en-1-yl]-N-(3,4-difluorophenyl)acetamide;
2-[3-(4-Bromophenyl)-2-oxo-7-oxa-1,4-diazaspiro[4.5]dec-3-en-1-yl]-N-[3-(trifluoromethyl)phenyl]acetamide; and
2-[3-(4-Bromophenyl)-2-oxo-7-oxa-1,4-diazaspiro[4.4]non-3-en-1-yl]-N-[3-(trifluoromethyl)phenyl]acetamide;
and salts thereof.

44. A method of treating psychosis which method comprises administering an effective amount of a compound as claimed in claim 27 to a patient in need thereof.

45. A method as claimed in claim 43 wherein the disorder is psychosis, including schizophrenia, dementia or attention deficit disorder.

46. A pharmaceutical composition comprising a compound as claimed in claim 27, and at least one pharmaceutically acceptable excipient.
